(19) United States
(12) Patent Application Publication
(10) Pub. No.: US 2026/0014246 A1
(43) Pub. Date: Jan. 15, 2026

(54) TREATMENT METHODS FOR VIRAL INFECTIONS

(71) Applicant: THE UAB RESEARCH FOUNDATION, Birmingham, AL (US)

(72) Inventors: G.M. Anantharamaiah, Hoover, AL (US); C. Roger White, Maylene, AL (US); Paul Wolkowicz, Hoover, AL (US); Mayakonda N. Palgunachari, Birmingham, AL (US); Kevin Scott Harrod, Birmingham, AL (US)

(21) Appl. No.: 18/992,545

(22) PCT Filed: Jul. 11, 2022
(86) PCT No.: PCT/US2022/036737

§ 371 Date: Jan. 8, 2025

(51) Int. Cl. A61K 39/215; A61K 38/10; A61K 39/00; A61K 39/145; A61K 45/06; A61P 31/16

(52) U.S. Cl. CPC A61K 39/215; A61K 38/10; A61K 39/145; A61K 45/06; A61P 31/16; A61K 2039/54; A61K 2039/545

(57) ABSTRACT

The current disclosure provides methods for the production of ‘vaccine-like’ antiviral preparations. The present disclosure provides compounds for the treatment of a wide range of enveloped viral diseases and conditions. The present disclosure further provides methods for treating, preventing, and/or suppressing an enveloped virus disease in a subject using the compounds disclosed herein as well as pharmaceutical compositions comprising such compound.

### BACKGROUND OF THE DISCLOSURE

Viral diseases cause a significant impact worldwide. While somewhat effective treatments are available for a handful of viruses such as human immunodeficiency virus (HIV), human cytomegalovirus (HCV), and the influenza virus, few treatments are available for most other viral diseases. For example, bronchiolitis (of which respiratory syncytial virus is a major cause) is the second leading cause of childhood death world-wide with no effective treatment currently being available. Further, new treatments are also needed for viruses that have current treatments for use when these current therapies become ineffective (as has been observed for anti-influenza medications over the years).

Antiviral medications are required to inhibit viral reproduction without harming the host. One difficulty in developing any anti-viral medication is that viruses rely in a large part on the machinery of the host cells to reproduce. Therefore, only few viral targets are available for antiviral drugs. In addition, there are a wide range of viral types which rely on distinct mechanisms for viral replication and transmission. Some viruses use RNA to store their genetic information, while others use DNA. Some viruses are surrounded by a membrane commonly known as an envelope, others are not. Focusing on the individual characteristics of this staggeringly wide range of viral types hampers the development of a treatment that targets common viral molecular structures to effectively eradicate viral disease. Of note here is the fact that viruses surrounded by a membrane envelope cause the preponderance of human and non-human viral diseases.

Current viral treatments include the following classes of compounds: i) viral DNA/RNA polymerase inhibitors; ii) reverse transcriptase inhibitors; iii) viral protease inhibitors; iv) viral neuraminidase inhibitors; v) receptor antagonists; vi) membrane fusion inhibitors; vii) immunomodulating compounds; and viii) vaccines.

However, these approaches suffer from the shortcoming that they are specific for one viral protein target, a single virus or a small subsets of viruses. In addition, rapid genetic mutation and viral evasion of the host immune response by ‘apoptotic cell mimicry’ may render treatments ineffective against some viruses over time.

The art is lacking a treatment that targets a large group of viruses via a common mechanism, a treatment that is less susceptible to being rendered ineffective by viral mutation and the like, and antiviral treatments that provide prolonged protection against these pathogens.

In prior art are found three methods to produce vaccine preparations that can protect humans and non-humans against enveloped viruses. The first method exploits live virus that is genetically attenuated to reduce or eliminate human infection. The oral poliomyelitis vaccine is one example of such a ‘live-attenuated’ virus. The second method uses virus that is inactivated chemically to make it non-functional. Here exhaustive treatment with agents such as detergents or cross-linking agents such as beta-propio-lactone are used to destroy and inactivate virus. The parenterally administered polio vaccine is an example of this ‘inactivated’ virus approach. The third method administers encapsulated mRNA to subjects. The mRNA is specific for the viral target. Cells in the subject accumulate this mRNA, translate it into protein which is presented by cells to the host immune system. This elicits a host immune response.

‘Live-attenuated virus’ vaccine preparations produce a robust systemic and mucosal immune response. This allows only one or just a few doses of orally administered vaccine to provide full, often life-long, protection against an enveloped virus and its variants. ‘Live-attenuated’ virus vaccines, however, have a significant limitation in that they may revert back to an active form that can infect humans and produce the disease the vaccine sought to prevent. This circumstance is the main reason that the effective oral poliomyelitis vaccine was discontinued.

The second ‘chemically inactivated virus’ vaccine preparations produce a much less robust systemic response compared to ‘live attenuated’ virus. As they are given parenterally, intra-muscularly as an example, these ‘inactivated virus’ vaccines require booster shots and provide little mucosal immunity. Further, the cross-linking/denaturation process used to inactivate virus destroy many antigenic sites on the virus, which may reduce immunogenicity.

The third ‘mRNA’ vaccine preparation is given parenterally. It elicits a less robust systemic response than ‘live attenuated’ virus vaccines. It also focuses this antigenic response on highly specific regions of select proteins with SARS-COV2 spike protein as an example. These antibodies are largely neutralizing ones. While useful, this specificity of antigenic response does not possess the therapeutic advantages inherent in a broadly neutralizing antibody response seen against some other human viral diseases such as HIV. This specificity may also cause loss of vaccine efficacy in the face of rapid viral mutation of the target mRNA sequence.

The current disclosure provides a fourth and unique method for the production of ‘vaccine-like’ preparatons whose development will produce medicaments that possess the advantages of earlier methods. Thus, the present disclosure provides a solution to the problems of the prior art by providing antiviral compounds, pharmaceutical compositions comprising such antiviral compounds, and methods of using such antiviral compounds and pharmaceutical compositions in the treatment of subjects. The disclosed antiviral compounds, pharmaceutical compositions, and methods target a wide range of viruses through a common mechanism which is resistant to the mechanisms of genetic drift and immune modulation.

Inactivation of enveloped viruses using the molecules claimed herein, or related compounds, will be irreversible. This attribute will overcome the problem of viral reactivation that can occur with genetic attenuation. Inactivators that target the viral envelope, as described in this disclosure, also will not obtund viral antigens as does the process of viral inactivation via chemical crosslinking. The method detailed within this disclosure will also produce a systemic immune response against a wide range of viral antigens offering the potential for a broadlny neutralizing response compared to the mRNA preparations.

Further, due to their unique mechanism of action, a single molecule of this disclosure, or a related compound, can inactivate many, probably all, enveloped viruses. This attribute of the method disclosed here will allow one or only a few of the molecules disclosed herein to generate ‘vaccine-like’ preparations against a wide range of enveloped viruses.

The preparation and method described in this disclosure identifies a ‘vaccine-like’ response which may occur through the classic immune cell paradigm or through the classic response in part coupled to a non-classic cytokine response. Either one or some variant of the second affords protection from exposure to enveloped virus.

These unique properties and mechanism of protection shall allow the method and the molecules described in this disclosure to eradicate enveloped virus diseases in human and non-human subjects.

### SUMMARY OF THE INVENTION

In some embodiments, the present disclosure relates to a composition comprising an effective amount of a peptide, or a pharmaceutically acceptable form thereof, wherein the peptide may form a cationic or zwitterionic amphipathic helix that may comprise an amino acid sequence defined from the group consisting of: SEQ ID NOS: 1-39. The composition may further comprise at least one other antiviral agent. In other embodiments, the present disclosure relates to compounds contain cationically charged and hydrophobic surfaces which mimic those defined in SEQ ID Nos: 1-39.

In some embodiments, the present disclosure relates to a method for treating an enveloped virus in a subject, comprising administering to said subject an effective amount of a peptide or a charged molecule, or a pharmaceutically acceptable form thereof, wherein the peptide or charged molecule contain cationic and hydrophobic regions that may form a cationic or zwitterionic amphipathic helix and may comprise a sequence selected from or charge distribution related to the group consisting of: SEQ ID NOS: 1-39.

In some embodiments of the method, the pharmaceutically acceptable form is a mixture or a medicament of peptide or charged molecule and enveloped virus in which the peptide/molecule inactivates the enveloped virus.

In some embodiments of the method this mixture is in saline. In some embodiments of the method the mixture is suspended in any other suitable carrier vehicle.

In some embodiments of the method, the subject is human. In some embodiments of the method, the subject is non-human including other mammals and birds. In some embodiments of the method, the subject is diagnosed with the viral infection. In some embodiments of the method, the subject is not infected with the enveloped virus. In some embodiments, the human subject is diagnosed with pulmonary disease, cardiovascular disease, diabetes mellitus, bacterial superinfection, sepsis syndrome, hypertension, chronic lung disease (inclusive of asthma, chronic obstructive pulmonary disease, and emphysema), chronic renal disease, chronic liver disease, immunodeficiency, an immunocompromised condition, neurologic disorder, neurodevelopmental, or intellectual disability.

In some embodiments of the method, the enveloped virus is selected from the group consisting of: Herpesviridae, Poxviridae, Hepadnaviridae, Asfarviridae, Flaviviridae, Togoviridae, Coronaviridae, Orthomyxoviridae, Paramyxoviridae, Rhabdoviridae, Bunyaviridae, Filoviridae, and Retroviridae. In some embodiments of the method, the enveloped virus is SARS-CoV-2. In some embodiments of the method, the enveloped virus is influenza.

In some embodiments of the method, the administration of the compound or pharmaceutically acceptable form thereof comprises two or more doses.

In some embodiments of the method, a disease or condition related to the enveloped virus is treated or prevented. In some embodiments of the method, the disease or condition is Guillain-Barre Syndrome, primary viral pneumonia, secondary bacterial pneumonia, inflammatory conditions, multisystem inflammatory syndrome, tissue damage, or organ failure, immune system over activation, or combinations of the foregoing.

In some embodiments of the method, the method further comprises administering at least one additional antiviral agent.

In some embodiments of the method, the administration is parenteral, pulmonary, intra-nasal, buccal, oral-gastric or intravenous.

In some embodiments of the method, after administration of the peptide or the charged molecule, the viral load of the enveloped virus is reduced in the subject.

In some embodiments of the method, administration of the peptide or a pharmaceutically acceptable form of [peptide-virus] complex prevents viral infection and inhibits increase in viral load in the subject.

In some embodiments of the method, the peptide or charged molecule is complexed to lipids prior to administration to the subject. In some embodiments of the method, these [peptide/charged molecule-lipid] complexes decrease viral load or reduce viral pathology similar to or better than the peptide/molecule or [peptide/molecule-virus] mixture themselves.

In some of the embodiments of the method the peptide or molecule are comprised of natural enantiomers while in other embodiments the peptide or molecule are comprised of atypical enantiomers.

### BRIEF DESCRIPTION OF THE FIGURES

FIG. 1A shows the antiviral activity of EVE5 in SARS-COV-2 infected Vero-E6 cells in vitro. FIG. 1B show a graphical representation of the results shown in FIG. 1A.

FIG. 2A shows the antiviral activity of EVEmR18L in SARS-COV-2 infected Vero-E6 cells in vitro. FIG. 2B show a graphical representation of the results shown in FIG. 2A.

FIG. 3A shows the antiviral activity of EVE4 in SARS-COV-2 infected Vero-E6 cells in vitro. FIG. 3B show a graphical representation of the results shown in FIG. 3A.

FIG. 4 shows a comparison of the antiviral activity of remdesivir as compared to EVE4, EVE5, and EVEmR18L in Vero-E6 cells in vitro.

FIG. 5A shows the antiviral activity of EVE4 in SARS-COV-2 infected Vero-E6 cells in vitro. FIG. 5B shows the antiviral activity of EVE5 in SARS-COV-2 infected Vero-E6 cells in vitro. FIG. 5C shows the antiviral activity of EVEmR18L in SARS-COV-2 infected Vero-E6 cells in vitro.

FIG. 6A shows the lack of cytotoxic effects of EVE4, EVE5, EVEmR18L and 4F in Vero-E6 cells in vitro after 24 hours incubation. FIG. 6B shows the lack of cytotoxic effects of EVE4, EVE5, EVEmR18L and 4F in Vero-E6 cells in vitro after 48 hours incubation. FIG. 6C shows the lack of cytotoxic effects of EVE4, EVE5, EVEmR18L and the 4F peptide as a [peptide-lipid] complex in Vero-E6 cells in vitro after 48 hours incubation.

FIG. 7 shows a helical wheel diagram of peptides EVEmR18L, EVEmR14L, and Rev-EVEmR14L. Hydrophobic amino acids are represented by bold circles and charged amino acid residues are indicated.

FIG. 8 shows a helical wheel diagram of peptides 2F-EVE14, 3F-EVE14 (EVE14), 3F-EVE18 and 4F-EVE18 (EVE18). Hydrophobic amino acids are represented by bold circles and charged amino acid residues are indicated.

FIG. 9A shows the suppression of weight loss that occurs with pre-incubation of 10 micromolar EVE14 with 1200 pfu H1N1 for 1, 5 and 60 minutes prior to intranasal administration of the [EVE14-H1N1] mixture to female C57BL/6J mice. FIG. 9B shows the increase in survival by pre-incubation of 10 micromolar EVE14 with 1200 pfu H1N1 prior to intranasal administration of the [EVE14-H1N1] mixture to female C57 mice.

FIG. 10 shows that little weight loss occurs in female C57-BL/6J mice given an intranasal [EVE14-H1N1] mixture that had been pre-incubated for 60 minutes. These mice show no weight loss when given a second challenge with active H1N1 (1200 pfu) three weeks later.

FIG. 11 shows the weight loss of three groups of female C57 mice given (i) 1200 pfu H1N1 intranasally, (ii) H1N1 intranasally followed by sub-cutaneous administration of 300 micrograms EVE14 2 hr after H1N1 and 150 micrograms EVE14 24 hr and 48 hr later, and (iii) H1N1 (1200 pfu) given intranasally followed by sub-cutaneous administration of 300 micrograms EVE14 2 hr later then 150 micrograms of EVE14 sub-cutaneously 24 hr, 48 hr, 72 hr and 96 hr after H1N1.

FIG. 12 shows the improvement in survival of the three groups of mice reported in FIG. 11.

FIG. 13 shows H1N1 viral load over time reported by others in mice with intranasal or intratracheal administration of virus.

FIG. 14 shows the weight loss and survival of two groups of female C57BL/6J mice. The first group was challenged intranasally with 1200 pfu H1N1 while the second group was challenged with H1N1 and then given 300 micrograms of EVE14 sub-cutaneously 2 hr later followed by 150 micrograms EVE14 subcutaneously 24 hr and 48 hr after virus. The initial bodyweight of all surviving mice is reported. After a two week observation period and a one week recovery period, all surviving mice were challenged intranasally with 1200 pfu of active H1N1. Both groups showed immune protection against a second challenge with active H1N1.

FIG. 15 shows that the EVE14 virolytic inactivates H1N1A-California making this EV incapable of causing death in female C57B1-6J mice.

FIG. 16 shows that the EVE18 virolytic inactivates H1N1A-California making this EV incapable of causing death in female C57BL-6J mice.

FIG. 17 shows that H1N1A-California inactivated with EVE18 when given intranasally to female C57-BL/6J mice protects them from death by a subsequent challenge with active H1N1.

FIG. 18 shows remdizivir offers only little protection against SARS-COV-2 Alpha in female hACE2 Knock-in mice.

FIG. 19 shows that EVE14 inactivates SARS-COV-2 Alpha making it incapable of causing death in female hACE2 Knock-in mice.

FIG. 20 shows that EVE18 inactivates SARS-COV-2 Alpha making it incapable of causing death in female hACE2 Knock-in mice.

FIG. 21 shows the intranasal administration of SARS-COV-2 Alpha treated with EVE18 to female hACE2 Knock-in mice protects them from death following a subsequent challenge with active SARS-COV-2 Alpha. That is, virolytics such as EVE18 protect against viral pathology in a homologous manner.

FIG. 22 shows that EVE18 inactivates SARS-COV-2 Delta making it incapable of causing death in female hACE2 Knock-in mice.

FIG. 23 shows that the intranasal administration of SARS-COV-2 Alpha treated with EVE18 to female hACE2 Knock-in mice protects them from death following a subsequent challenge with active SARS-COV-2 Delta. That is, virolytics like EVE18 protect against viral pathology in a heterologous manner.

FIG. 24 shows that therapeutic dosing of EVE18 decreases mortality in female hACE2 Knock-in mice challenged with SARS-COV-2 Alpha.

### DETAILED DESCRIPTION

The present disclosure provides compounds for the treatment of a wide range of viral diseases and conditions. The present disclosure further provides methods for treating, preventing, and/or suppressing a viral disease in a subject using the compounds disclosed herein as well as pharmaceutical compositions comprising such compounds.

### Definitions

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The articles “a” and “an” are used herein to refer to one or to more than one (i.e., to at least one) of the grammatical object of the article. By way of example, “an element” means one element or more than one element.

The term “cellular entry” as used herein refers to the process through which a virus gains entry into a cell of a subject to initiate a viral infection in the cell. As an example, with SARS-CoV-2 such process involves binding of a viral spike protein to a host cell protein or component that acts as an entry receptor for the virus (ACE2). The term “cellular entry” may also include ancillary events required for viral binding to a host cell receptor, such as enzymatic processing of a viral and or host cell protein.

The term “compound(s) of the disclosure” as used herein refers to a polypeptide disclosed herein, suitably one or more of a cationic or zwitterionic amphipathic peptide including EVE4, EVE5, EVE14 or EVE18, or to a compound which has the ionic charge distribution and surface properties of such peptides. A compound of the disclosure may be present in any pharmaceutically or therapeutically acceptable form.

The term an “effective amount,” “sufficient amount” or “therapeutically effective amount” as used herein is an amount of a compound that is sufficient to achieve a beneficial or desired result, including but not limited to clinical results. As such, the effective amount may be sufficient, for example, to reduce or ameliorate the severity and/or duration of a viral infection, such as but not limited to, a SARS-COV-2 infection, or one or more symptoms thereof, prevent the recurrence, development, or onset of one or more symptoms associated with the viral infection, prevent or reduce the replication or multiplication of a virus, prevent or reduce the production and/or release of a viral particle, or enhance or otherwise improve the prophylactic or therapeutic effect(s) of another therapy. In certain embodiments, an effective amount is an amount of the compound of the disclosure that avoids or substantially attenuates undesirable side effects.

In certain embodiments, the “effective amount,” “sufficient amount” or “therapeutically effective amount” in the context of a viral infection is an amount sufficient to reduce viral replication in the subject or reduce viral cellular entry in a subject. Such a reduction in any of the foregoing may be by at least 5%, preferably at least 10%, at least 15%, at least 20%, at least 25%, at least 30%, at least 35%, at least 40%, at least 45%, at least 50%, at least 55%, at least 60%, at 100%

In some embodiments, the “effective amount,” “sufficient amount” or “therapeutically effective amount” in the context of a viral infection increases the survival rate of infected subjects by at least 5%, preferably at least 10%, at least 15%, at least 20%, at least 25%, at least 30%, at least 35%, at least 40%, at least 45%, at least 50%, at least 55%, at least 60%, at least 65%, at least
70%, at least 75%, at least 80%, at least 85%, at least 90%, at least 95%, or 100%. In each of the foregoing, when a reduction of increase is specified, such reduction of increase may be determined with respect to a subject that has not been treated with a compound of the disclosure and that has a diagnosed viral infection of the same type.

The term “excipient” as used herein means a substance formulated alongside the active ingredient of a medication included as a carrier, for the purpose of long-term stabilization, bulking up solid formulations that contain potent active ingredients in small amounts (thus often referred to as bulking agents, fillers, or diluents), or to confer a therapeutic enhancement on the active ingredient in the final dosage form, such as facilitating drug absorption, reducing viscosity, aiding drug-virus complex survival in acidic and/or basic conditions, enhancing solubility or to render the active ingredient palatable or consumable by the subject either human or non-human. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. Suitable pharmaceutical excipients are described in “Remington: The Science and Practice of Pharmacy”, Academic Press, Hardcover ISBN: 9780128200070, Editor Hardcover ISBN: 9780128200070, 23^{rd} Edition. Pharmaceutical Sciences” by E. W. Martin.

The term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

The term “pharmaceutically acceptable form” is meant to include known forms of a compound that may be administered to a subject, including, but not limited to, solvates, hydrates, prodrugs, isomorphs, polymorphs, pseudomorphs, neutral forms and salt forms of a compound.

The term “pharmaceutically acceptable form” is meant to include formulations of peptide or compound mixtures with one or more enveloped virus along with the other ingredients noted in the preceding paragraphs. These mixtures produce a medicament whose administration to a patient subject or to livestock subject reduces symptoms, disease infectivity or viral load.

The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic,
citric, methanesulfonic, benzenesulfonic, benzoic, malonic, trifluoroacetic, trichloroacetic, naphthalene-2 sulfonic and other acids. Pharmaceutically acceptable salt forms may also include forms wherein the ratio of molecules comprising the salt is not 1:1. For example, the salt may comprise more than one inorganic or organic acid molecule per molecule of base, such as two hydrochloric acid molecules per molecule of the compounds of this disclosure. As another example, the salt may comprise less than one inorganic or organic acid molecule per molecule of base, such as two molecules of compound of this disclosure per molecule of tartaric acid. Salts may also exist as solvates or hydrates.

A “pharmaceutical composition” refers to a mixture of one or more of the compounds of the disclosure, with other components, such as, but not limited to, pharmaceutically and therapeutically acceptable excipients. The purpose of a pharmaceutical composition is to facilitate administration of a compound of disclosure or a therapeutically active mixture of compounds in this disclosure and viral particles

The term “solvate” as used herein means a compound of the disclosure, or a pharmaceutically acceptable salt thereof, wherein one or more molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

The terms “subject” and “patient” as used herein include all members of the animal kingdom including, but not limited to, mammals, animals (e.g., cats, dogs, horses, swine, etc.), birds and humans. In certain embodiments, the subject is a human.

The term, “Treatment” as used herein means an approach for obtaining beneficial or desired results, including but not limited to clinical results. For example, “Treatment” may be an approach to obtain a beneficial economic result wherein the mortality or pathology of a non-human species is improved by administration of one or more of the antiviral preparations described herein. Beneficial or desired clinical or other kind of result in the context of a viral infection include, but are not limited to, alleviation or amelioration of one or more symptoms or conditions of viral infection, a diminution of the extent of disease, a stabilized (i.e., not worsening) state of disease, preventing the spread of disease, delay or slowing of disease progression, amelioration or palliation of the disease state and remission (whether partial or total), whether detectable or undetectable. “Treatment” can also mean prolonging survival as compared to expected survival if not receiving treatment.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Compounds of the Disclosure

The present disclosure provides a family of small molecules capable of disrupting viral stability, integrity, and cell entry. Compounds of the disclosure inhibit or decrease the infectivity of target enveloped viruses. The present application demonstrates that the compounds of the disclosure are as or more effective than remdesivir in suppressing SARS-COV-2 infection in vitro. Furthermore, their biologically-derived structures can be easily modified to identify additional ionically charged compounds with enhanced potency and/or bio-availability.

Many prior art compounds, for example remdesivir, target specific proteins expressed in specific viruses (viral RNA polymerase in the case of remdesivir). The compounds of the disclosure are designed to target the viral envelope. As such, the compounds of the disclosure have the potential to show broad antiviral activity against a range of pathogenic viruses.

The viral envelope is the outermost layer of many types of viruses. The viral envelope protects the viral genetic material during the virus life-cycle. The envelopes are typically derived from portions of the host cell membranes (phospholipids and proteins) and generally includes viral-specific glycoproteins. Glycoproteins on the surface of the envelope serve to identify and bind to receptor sites on the host's membrane. The viral envelope may then fuse with the host's membrane, allowing the capsid and viral genome to enter and infect the host or the virus may internalize into cells by other mechanisms. Some enveloped viruses also have a capsid, another protein layer, between the envelope and the genome. The lipid bilayer envelope of these viruses is relatively sensitive to desiccation, heat, and detergents. Enveloped viruses possess great adaptability and can mutate their genetic material in a short time in order to create mutant proteins that evade the host immune system and can cause persistent infections for example Long COVID.

The viral envelope is common to all pathogenic enveloped viruses. As the viral envelope is acquired from host cells, viruses cannot maintain or repair their envelope, meaning disruption of the membrane envelope is irreversible. The viral envelope encapsulates the viral RNA/DNA, protects the viral DNA/RNA from non-viral nucleases, acts as a platform for capsid proteins, and is required for viral integrity and viral cellular entry. The viral envelope has been viewed as a passive player in the viral life cycle. Hence it has not been targeted by previous therapies. The present disclosure provides an alternative view of the viral envelope as a drug target and the compounds of the disclosure target and disrupt the viral envelope to inhibit viral infection.

Since the viral envelope derives from host cell membranes, the viral envelope contains an inner and an outer leaflet. The different types of membrane phospholipids distribute across these leflets in an asymmetric manner. To maintain this asymmetry requires bioenergetic input. Since enveloped viruses have no such bioenergetic ‘machinery’, phospholipid asymmetry does not persist in the viral envelope during the viral life-cycle.

One important consequence of this lack of phospholipid asymmetry is the following. Under energized conditions, the negatively charged phosphotidylserine phospholipid is contained within the inner, non-accessible membrane leaflet. Upon cell or membrane de-energization inner leaflet phosphatidylserine ‘flips’ to the outer, accessible membrane leaflet and introduces a significant negative charge to this surface. Importantly, such a negatively charged accessible surface occurs in apoptotic, dying cells. It is recognized by normal cells and immune cells as a ‘death’ signal which provokes systemic clearance of such negatively charged membranes.

Consqeuntly, all enveloped viruses possess an outer accessible membrane leaflet which has a high content of negatively charged phospholipids. This highly negatively charged outer membrane leaflet is a type of ‘viral apoptotic mimicry’ which tricks normal cells into internalizing enveloped virus to promote viral infection.

This type of ‘viral apoptotic mimicry’, which is common to all enveloped viruses, has not been targeted by antiviral medicaments.

Following treatment the cationic or zwitterionic amphipathic helical peptides, a cationic domain covalently attached to amphipathic helical peptide or a compound with distinct cationic and hydrophobic surfaces claimed in this application, will bind to and disrupt the functional integrity of the negatively charged outer membrane leaflet of all enveloped viruses. This causes the enveloped virus to enter a state that cannot effectively induce pathology but can still raise an immunological response in the subject. This state is analogous to bacterial ghost particles and are termed here ‘enveloped virus ghost particles.’ These ghost particles can be fully vesiculated viral envelope membrane or partially disrupted viral envelope membrane. Compounds claimed here can treat enveloped virus therapeutically as a medicament or can be mixed with the isolated enveloped virus to produce immunologically active viral ghosts. Administration of such ghost particles either as crude or as purified preparations to the subject prior to exposure to an active enveloped virus in question can raise an immune response which protects the subject from pathology following subsequent exposure to the active virus. Enveloped virus ghost particle purification can be obtained by any of several methods known to those practiced in the art. These methods include but are not limited to centrifugation or affinity separation. This administration of [enveloped virus-peptide/charged molecule] can be by a parenteral route, by an intranasal route, or by a buccal/oral-gastric route among others. Such a general approach is typical for vaccines produced in prior art using ‘live-attenuated’ virus or ‘chemically inactivated/cross-linked’ virus; for example, the vaccines against poliomyelitis virus. Since the peptide/charged molecule inactivation of enveloped viruses acts at their membrane, they do not denature enveloped virus proteins. Thus, the treatments and molecules revealed in this disclosure will produce both homologous and heterologous protection against enveloped viral infection. In the case of homologous protection, subjects will be protected against exposure to the active enveloped virus which was contained in the medicament. In the case of heterologous protection, the subject will be protected against infection with additional active enveloped viruses. Here, the host antibodies induced by the enveloped virus in the medicament will recognize and neutralize the proteins present in enveloped viruses that are structurally related to these proteins but are found in viruses not present in the medicament; for example, a [SARS-COV-2-Alpha-peptide] medicament may protect against infection by the SARS-CoV-2 Delta virus.

Pathogenic enveloped viruses include both DNA and RNA viruses. In one embodiment, the pathogenic enveloped virus is selected from the following viral families: Herpesviridae, Poxviridae, Hepadnaviridae, Asfarviridae, Flaviviridae, Togoviridae, Coronaviridae, Orthomyxoviridae, Paramyxoviridae, Rhabdoviridae, Bunyaviridae, Filoviridae, and Retroviridae. In one embodiment, the pathogenic enveloped virus is from the Coronaviridae family. In one embodiment, the pathogenic enveloped virus is SARS, SARS-COV-2, or MERS. In one embodiment, the pathogenic enveloped virus is SARS-COV-2.

Direct enveloped viral infection of cells is a major cause of the diseases associated with these pathogens. In addition, however, a localized or systemic inflammatory response can accompany viral infection or participate greatly in the post-infection period of the infected subject. A typical case of such an ‘inflammatory’ response to enveloped viral infection is the ‘Long COVID’ syndrome. In Long COVID, heart inflammation, kidney dysfunction and brain damage all associate with but also can arise following the initial SARS-COV2 infection. A prolonged inflammatory response in these organs is thought to provoke these ancillary but equally damaging post-infection disorders.

The peptides disclosed herein have well-documented anti-inflammatory properties that ameliorate the inflammation associated with a variety of agents including oxidized lipoproteins and bacterial lipopolysaccharides (e.g., Do, R. et al. Common variants associated with plasma triglycerides and risk for coronary artery disease. Nature Genetics 45:1345-1352 (2013)). These dual antiviral and anti-inflammatory properties of the disclosed peptides allow their therapeutic use both acutely, where enveloped virus must be destroyed and viremia reduced, and chronically, where low levels of residual enveloped virus and/or a prolonged inflammatory response to infection provokes sustained and destructive inflammation.

In one embodiment, the compounds of the disclosure comprise peptide compounds. In one embodiment, a compound of the disclosure is selected from the peptide compounds below.

EVE4 (AEM-28):
(SEQ ID NO: 1)
Ac-LRKLRKRLLRDWLKAFYDKVAEKLKEAF-NH₂;
EVE5 (AEM-2):
(SEQ ID NO: 2)
Ac-Aha-LRRLRRRLLRDWLKAFYDKVAEKLKEAF-NH₂;
EVEmR18L (mR18L):
(SEQ ID NO: 3)
Ac-GFRRFLGSWARIYRAFVG-NH₂;
Gly-EVEmR18L:
(SEQ ID NO: 4)
Ac-Gly-GFRRFLGSWARIYRAFVG-NH₂;
Ala-3F-EVEmR18L:
(SEQ ID NO: 5)
Ac-3F-Ala-GFRRFLGSWARIYRAFVG-NH₂;
Aha-EVEmR18L:
(SEQ ID NO: 6)
Ac-Aha-GFRRFLGSWARIYRAFVG-NH₂;
Rev-EVEmR18L:
(SEQ ID NO: 7)
Ac-GVFARYIRAWSGLFRRGF-NH₂;
Gly-Rev-EVEmR18L:
(SEQ ID NO: 8)
Ac-Gly-GVFARYIRAWSGLFRRGF-NH₂;
Ala-3F-Rev-EVEmR18L:
(SEQ ID NO: 9)
Ac-3F-Ala-GVFARYIRAWSGLFRRGF-NH₂;
Aha-Rev-EVEmR18L:
(SEQ ID NO: 10)
Ac-Aha-GVFARYIRAWSGLFRRGF-NH₂;
EVEmR18L (G⁷ to R):
(SEQ ID NO: 11)
Ac-GFRRFLRSWARIYRAFVG-NH₂;
EVEmR18L (G⁷ to K):
(SEQ ID NO: 12)
Ac-GFRRFLKSWARIYRAFVG-NH₂;
EVEmR18L (G⁷ to H):
(SEQ ID NO: 13)
Ac-GFRRFLHSWARIYRAFVG-NH₂;
EVEmR18L (G⁷ to E):
(SEQ ID NO: 14)
Ac-GFRRFLESWARIYRAFVG-NH₂;
EVEmR18L (G⁷ to A):
(SEQ ID NO: 15)
Ac-GFRRFLASWARIYRAFVG-NH₂;
EVEmR14L (mR14L):
(SEQ ID NO: 16)
Ac-RRFLGSWARIYRAF-NH₂;
EVEmR14L (G⁵ to K):
(SEQ ID NO: 17)
Ac-RRFLKSWARIYRAF-NH₂;
EVEmR14L (G⁵ to R):
(SEQ ID NO: 18)
Ac-RRFLRSWARIYRAF-NH₂;
EVEmR14L (G⁵ to H):
(SEQ ID NO: 19)
Ac-RRFLHSWARIYRAF-NH₂;
EVEmR14L (G⁵ to E):
(SEQ ID NO: 20)
Ac-RRFLESWARIYRAF-NH₂;
EVEmR14L (G⁵ to A):
(SEQ ID NO: 21)
Ac-RRFLASWARIYRAF-NH₂
Rev-EVEmR14L:
(SEQ ID NO: 22)
Ac-FARYIRAWSGLFRR-NH₂;
Rev-EVEmR14L (G¹⁰ to K):
(SEQ ID NO: 23)
Ac-FARYIRAWSKLFRR-NH₂;
Rev-EVEmR14L (G¹⁰ to R):
(SEQ ID NO: 24)
Ac-FARYIRAWSRLFRR-NH₂;
Rev-EVEmR14L (G¹⁰ to H):
(SEQ ID NO: 25)
Ac-FARYIRAWSHLFRR-NH₂;
Rev-EVEmR14L (G¹⁰ to E):
(SEQ ID NO: 26)
Ac-FARYIRAWSELFRR-NH₂;
Rev-EVEmR14L (G¹⁰ to A):
(SEQ ID NO: 27)
Ac-FARYIRAWSELFRR-NH₂;
2F-EVEmR14L:
(SEQ ID NO: 28)
Ac-RRFLGSWARIYRAF-NH₂;
3F-EVE14 (EVE14):
(SEQ ID NO: 29)
Ac-RRFYGSIWRFIRAF-NH₂;
Rev-3F-EVE14:
(SEQ ID NO: 30)
Ac-FARIFRWISGYFRR-NH₂;
3F-EVEmR18L:
(SEQ ID NO: 31)
Ac-GFRRFLGSWARIYRAFVG-NH₂;
4F-EVE18 (EVE18):
(SEQ ID NO: 32)
Ac-GFRRFYGSIWRFIRAFVG-NH₂;
Rev-4F-EVE18:
(SEQ ID NO: 33)
Ac-GVFARIFRWISGYFRRFG-NH₂;
4F:
(SEQ ID NO: 34)
Ac-DWFKAFYDKVAEKFKEA-NH₂;
Rev-4F:
(SEQ ID NO: 35)
Ac-AEKFKEAVKDYFAKFWD-NH₂;
4F, 2L-EVE18:
(SEQ ID NO: 36)
Ac-GFRRFYGSLWFRLRAFVG-NH₂;
Rev-4F, 2L-EVE18:
(SEQ ID NO: 37)
Ac-GVFARLRFWLSGYFRRFG-NH₂;
3F, 2L-EVE14:
(SEQ ID NO: 38)
Ac-RRFYGSLWFRLRAF-NH₂;
Rev-3F, 2L-EVE14:
(SEQ ID NO: 39)
Ac-FARLRFWLSGYFRR-NH₂.

In the structures claimed in SEQ ID NO. 1-39 the acetyl groups (Ac) can be replaced by other aliphatic groups including as an example butyrate. These groups may range from 1 to 18 carbons.

The arginine residues (R) in these structures may be replaced by other cationic residues such as but not limited to lysine (K). The lysine side chain amine can itself be modified with aliphatic compounds of increasing length from 1 to 18 carbons. The cationic lysine charge should be maintained, for example lysine to dimethyl lysine.

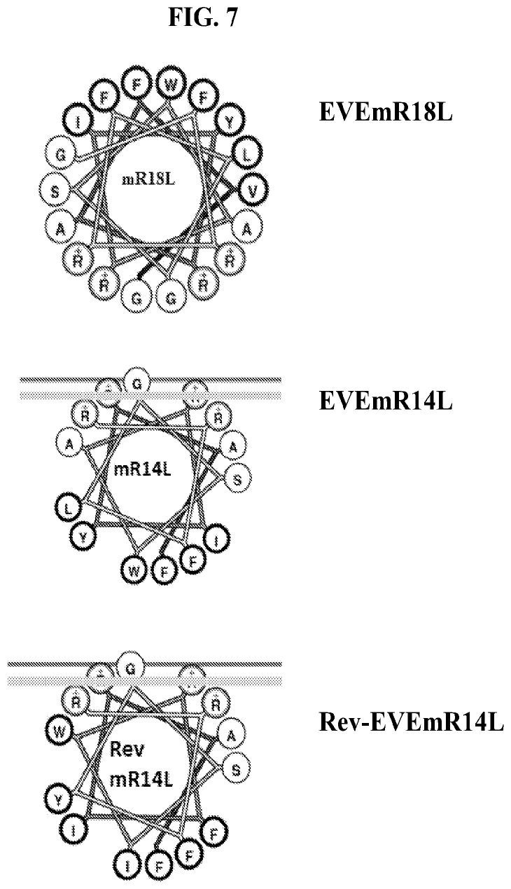

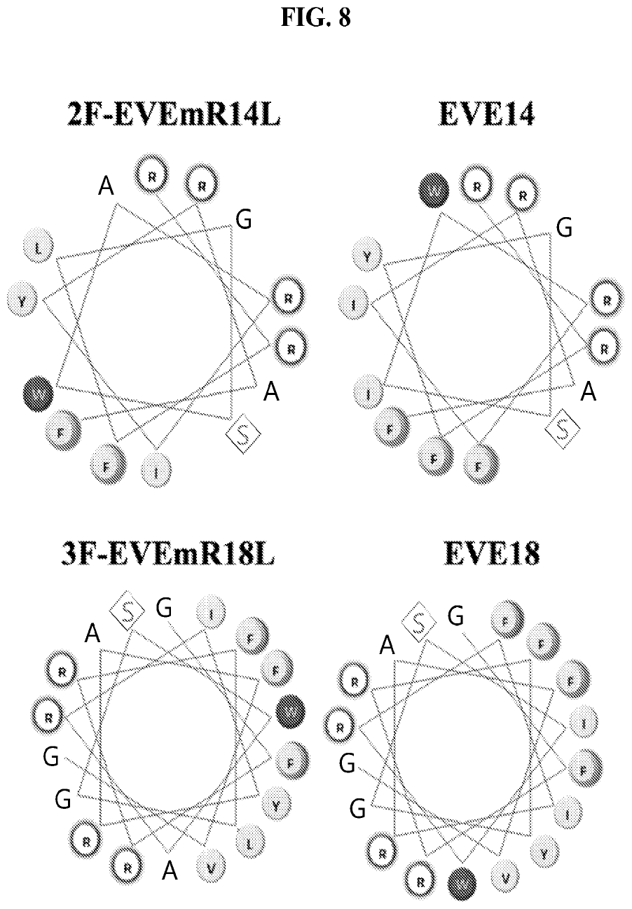

A helical wheel for peptides EVEmR18L, EVEmR14L, and Rev-EVEmR14L is shown in FIG. 7. A helical wheel for peptides 2F-EVEmR14L (Upper left), 3F-EVE14 (EVE14: Upper right)), 3F-EVEmR18L (Lower left) and 4F-EVE18 (EVE18: Lower right) is shown in FIG. 8.

In other embodiments, the compounds comprise cationic or zwitterionic peptides that may form amphipathic helices.

In other embodiments, the compounds comprise neutral or charged membrane-active compounds including but not limited to the Tween, the Triton, and the octylglucoside series of detergents or chemical combinations of peptides and such membrane-active agents.

In other embodiments, the compounds of the disclosure are comprised of the L-enantiomers of the constituent amino acids.

In other embodiments, the compounds of the disclosure are comprised of the D-enantiomers of the constituent amino acids.

In other embodiments, the compounds may consist of mostly L-enantiomers with one or more D-enantiomers inserted into the sequence of the constituent amino acids.

### Methods of Treatment

The present disclosure provides methods for the treatment and/or prevention of a viral infection in a subject, the method comprising administering a therapeutically effective amount of a compound or a crude or purified [compound-enveloped virus] mixture of the disclosure to the subject.

In a first aspect, the disclosure provides a method for treating a subject suffering from a viral infection, the method comprising administering to said subject an effective amount of a compound of the disclosure, or a pharmaceutically acceptable form thereof, either alone or as a part of a pharmaceutical composition.

In a second aspect, the disclosure provides a method for suppressing a viral infection in a subject, the method comprising administering to the subject an effective amount of a compound of the disclosure, or a pharmaceutically acceptable form thereof, either alone or as a part of a pharmaceutical composition or mixture.

In a third aspect, the disclosure provides a method for preventing a viral infection in a subject, the method comprising administering to the subject an effective amount of a compound of the disclosure, either alone or as a part of a pharmaceutical composition or mixture.

In a fourth aspect, the disclosure provides a method for treating, suppressing and/or preventing a disease or condition relating to viral infection, the method comprising administering to said subject an effective amount of a compound of the disclosure, or a pharmaceutically acceptable form thereof, either alone or as a part of a pharmaceutical composition or mixture. In certain embodiments of the fourth aspect, the disease or condition is Guillain-Barre Syndrome, primary viral pneumonia, secondary bacterial pneumonia, inflammatory conditions, multisystem inflammatory syndrome, tissue damage, or organ failure, immune system over activation, or combinations of the foregoing.

In a fifth aspect, the disclosure provides a method for reducing viral titer of a virus in a bodily fluid, tissue or cell of a subject, the method comprising administering an effective amount of a compound of the disclosure, or a pharmaceutically acceptable form thereof, to the subject, either alone or as a part of a pharmaceutical composition. In certain embodiments, the transmission of the virus (for example, from a subject infected with the virus to a subject that is not yet infected) is reduced as a result of the reduced viral titer.

In a sixth aspect, the disclosure provides a method for reducing or preventing the transmission of a viral infection from a first subject to a second subject, the method comprising administering to the first subject an effective amount of a compound of the disclosure or a pharmaceutically acceptable form thereof, either alone or as a part of a pharmaceutical composition or mixture. In certain embodiments of the sixth aspect, such reduction or prevention is obtained, at least in part, by reducing the cellular entry of a virus in the first subject. In certain embodiments, the compound of the disclosure is administered to the first subject before the first subject has been infected with the virus, after the first subject has been infected with the virus, or after the first subject has been infected with the virus and before the viral infection can be detected.

In a seventh aspect, the disclosure provides a method for reducing or preventing the transmission of a viral infection from a first subject to a second subject, the method comprising administering to the second subject an effective amount of a compound of the disclosure, or a pharmaceutically acceptable form thereof, either alone or as a part of a pharmaceutical composition or mixture. In certain embodiments of the seventh aspect, the second subject may be at risk for viral infection. In certain embodiments of the seventh aspect, such reduction or prevention is obtained, at least in part, by preventing or reducing virus cellular entry in the second subject. In certain embodiments of the seventh aspect, such reduction or prevention is obtained, at least in part, by preventing or suppressing a viral infection in the second subject. In certain embodiments of the seventh aspect, such reduction or prevention is obtained by inducing in the second subject a protective immune response that arises from administering a pharmacologically acceptable composition or mixture of a compound of the disclosure and the virus to the second subject before their exposure to the virus. In one aspect of this embodiment, if a viral infection occurs in the second subject, it can be eliminated physiologically (for example, by the immune system) by the second subject, either with or without the administration of additional therapeutic compounds. In certain embodiments of the seventh aspect, the compound of the disclosure is administered to the second subject before the second subject has been infected with the virus.

In an eighth aspect, the disclosure provides a method of inhibiting infection of a cell of a subject with a virus, the method comprising administering to the first subject an effective amount of a compound of the disclosure, or a pharmaceutically acceptable form thereof, either alone or as a part of a pharmaceutical composition. In certain embodiments of the eighth aspect, the compounds of the disclosure inhibit the formation of the viral envelope and/or stimulating the degradation of the viral envelope after formation.

In a ninth aspect, the disclosure provides a method of inhibiting the formation of the viral envelope of a virus and/or stimulating the degradation of the viral envelope of a virus, the method comprising administering to the first subject an effective amount of a compound of the disclosure, or a pharmaceutically acceptable form thereof, either alone or as a part of a pharmaceutical composition.

In a tenth aspect, the disclosure provides a method of introducing at least one additional antiviral agent such as an enzyme that degrades viral RNA or DNA. In this embodiment, the peptide disrupts enveloped virus membrane integrity to allow access of nucleic acid degrading enzymes or other antiviral agents to their targets which had been previously denied by the intact enveloped virus membrane. This tenth aspect also includes any method of purifying (i) viral envelope membrane or (ii) envelope membrane vesicles that arise following the incubation of virus with the compounds of this disclosure. This method removes any residual active virus so as to create a fully non-pathogenic medicament.

The methods of the first to tenth aspects may further comprise one or more of the steps: (i) identifying a subject in need or treatment, prevention, suppression, reduction, or inhibition; and (ii) providing a compound of the disclosure or a pharmaceutical composition comprising a compound of the disclosure or a compound-virus mixture.

In the embodiments of the first to tenth aspects, the enveloped virus may be any member of the family Herpesviridae, Poxviridae, Hepadnaviridae, Asfarviridae, Flaviviridae, Togoviridae, Coronaviridae, Orthomyxoviridae, Paramyxoviridae, Rhabdoviridae, Bunyaviridae, Filoviridae, and Retroviridae. In the embodiments of the first to tenth aspects, the enveloped virus may be any member of the family Asfarviridae, Flaviviridae, Coronaviridae, Orthomyxoviridae, Hepadnaviridae, and Paramyxoviridae. In the embodiments of the first to tenth aspects, the enveloped virus may be any member of the family Coronaviridae capable of infecting a subject, including but not limited to a human subject. In any of the methods of the first to tenth aspects, the enveloped virus is a member of the genus Betacoronavirus, preferably but not exclusively capable of infecting a human subject. In any of the methods of the first to tenth aspects, the enveloped virus is a member of the genus Alphacoronavirus, preferably but not exclusively capable of infecting a human subject. In any of the methods of the first to tenth aspects, the virus is a member of the genus Betacoronavirus and the subgenus Embecovirus, Sarbecovirus or Merbecovirus, preferably but not exclusively capable of infecting a human subject. In any of the methods of the first to tenth aspects, the enveloped virus is a member of the genus Alphacoronavirus and the subgenus Dovinacovirus or Setracovirus, preferably but not exclusively capable of infecting a human subject.

In any of the methods of the first to tenth aspects, the enveloped virus is SARS-COV, SARS-COV-2, MERS-COV, HCOV-229E, HCoV-OC43, HCoV-NL63, HCoV-HKU1, respiratory syncytial virus, African swine fever virus, dengue virus, influenza viruses, human immunodeficiency virus, or hepatitis C virus or other representatives of this type of virus. In any of the methods of the first to tenth aspects, the virus is respiratory syncytial virus, African swine fever virus, dengue virus, influenza viruses, human immunodeficiency virus, or hepatitis C virus. In any of the methods of the first to tenth aspects, the virus is SARS-COV, SARS-COV-2, or MERS-COV. In any of the methods of the first to tenth aspects, the virus is SARS-COV. In any of the methods of the first to tenth aspects, the virus is SARS-COV-2. In any of the methods of the first to tenth aspects, the virus is MERS-COV.

In any of the methods of the first to tenth aspects, the compound or mixture of the disclosure may be used in combination with other antiviral agents, such as inhibitors of viral RNA polymerase activity and/or serine and non-serine protease inhibitors.

In any of the methods of the first to tenth aspects, the compound or mixture of the disclosure may be used in combination with agents that specifically degrade RNA or DNA including those polynucleotides found in virus.

In any of the methods of the first to tenth aspects, the viral infection comprises infection by a member of the family Coronaviridae and/or by one or more additional viruses.

In any of the methods of the first to tenth aspects, the compound of the disclosure may be administered in an effective amount. Suitable effective amounts are described in more detail herein. In any of the methods of the first to tenth aspects, the administering step may comprise administering a single dose of a compound of the disclosure according to a course of treatment (where the dose may contain an effective amount of a compound of the disclosure).

In any of the methods of the first to tenth aspects, the administering step may comprise administering more than one dose of a compound of the disclosure according to a course of treatment (where one or more doses may contain an effective amount of a compound of the disclosure). The amount of a compound of the disclosure in each dose administered during a course of treatment is not required to be the same. For example, in any of the methods of the first to tenth aspects, the administering step may comprise administering at least one loading dose and at least one maintenance dose during a course of treatment. Examples of dosing are described in more details herein.

In one embodiment of the first to tenth aspects, the administering step comprises administering a dose or a plurality of doses comprising a compound of the disclosure according to a course of treatment.

In another embodiment of the first to tenth aspects, the administering step comprises administering at least one loading dose and a plurality of maintenance doses comprising a compound of the disclosure via the respiratory tract according to a course of treatment. Dosing is described in more details herein.

In one embodiment of the first to tenth aspects, the administering steps comprise administering a dose of compound or compound-virus mixture parenterally (intramuscular dosing as an example) or buccally/orally-gastrically.

In any of the methods of the first to tenth aspects, a pharmaceutical composition and/or medicaments comprising a compound of the disclosure may be administered according to the methods described herein.

In any of the methods of the first to tenth aspects, a pharmaceutical composition and/or medicament comprising a compound or a compound-virus mixture of the disclosure may be administered in the food or a [food-medicament] mixture for consumption by human subjects, domesticated or non-domesticated animal or bird subjects.

In any of the methods of the first to tenth aspects, the subject is a mammal. In any of the methods of the first to tenth aspects, the subject is a human. In any of the methods of the first to tenth aspects, the subject is a bat or other carriers of enveloped viruses capable of infecting humans as known to any versed in this art. In any of the methods of the first to tenth aspects, the subject is avian. In any of the methods of the first to tenth aspects, the subject is a swine or pig. In any of the methods of the first to tenth aspects, the subject is a cow or a bull.

In any of the methods of the first to tenth aspects, the administering step may occur before the subject has been infected with an enveloped virus (i.e., the subject is at risk for infection), after the subject has been infected with an enveloped virus (but before an infection can be detected), or after a subject has been infected with an enveloped virus and before the infection can be detected.

In any of the methods of the first to tenth aspects, the subject is a healthcare worker, a first responder (for example, a policeman or a fireman), or a member of the military as such individuals may be required to undertake activities that place them at a higher risk of viral infection.

In any of the methods of the first to tenth aspects, the subject has travelled to a region where viral infections have been documented, the subject has had contact with a person who has travelled to a region where viral infections have been documented, the has had contact with a person who has a viral infection (including a viral infection that has not been detected) or is suspected of having a viral infection, the subject is a family member or acquaintance of a person who has a viral infection (including a viral infection that has not been detected) or is at risk of having a viral infection, the subject is an infant or child (for example, a subject under the age of 16 years) who has a caregiver or parent who has a viral infection or is at risk of having a viral infection.

In any of the methods of the first to tenth aspects, the subject may be suffering from pulmonary disease, cardiovascular disease, diabetes mellitus, bacterial superinfection, sepsis syndrome, hypertension, chronic lung disease (inclusive of asthma, chronic obstructive pulmonary disease, and emphysema), chronic renal disease, chronic liver disease, immunodeficiency, an immunocompromised condition, neurologic disorder, neurodevelopmental, or intellectual disability.

In any of the methods of the first to tenth aspects, a compound of the disclosure is EVE4, or a pharmaceutically acceptable form thereof. In any of the methods of the first to tenth aspects, a compound of the disclosure is EVE5, or a pharmaceutically acceptable form thereof. In any of the following methods of the first to tenth aspects, the compound can be either EVE18 or EVEmR18L with equivalence. In any of the methods of the first to tenth aspects, a compound of the disclosure is EVE18 (or, for example EVEmR18L), or a pharmaceutically acceptable form thereof. In any of the methods of the first to tenth aspects, a compound of the disclosure is Gly-EVE18, or a pharmaceutically acceptable form thereof. In any of the methods of the first to tenth aspects, a compound of the disclosure is 3F-Ala-EVE18, or a pharmaceutically acceptable form thereof. In any of the methods of the first to tenth aspects, a compound of the disclosure is Aha-EVE18, or a pharmaceutically acceptable form thereof. In any of the methods of the first to tenth aspects, a compound of the disclosure is Rev-EVE18, or a pharmaceutically acceptable form thereof. In any of the methods of the first to tenth aspects, a compound of the disclosure is Gly-Rev-EVE18, or a pharmaceutically acceptable form thereof. In any of the methods of the first to tenth aspects, a compound of the disclosure is 3F-Ala-Rev-EVE18, or a pharmaceutically acceptable form thereof. In any of the methods of the first to tenth aspects, a compound of the disclosure is Aha-Rev-EVE18, or a pharmaceutically acceptable form thereof. In any of the methods of the first to tenth aspects, a compound of the disclosure is EVE18 (G⁷ to R), or a pharmaceutically acceptable form thereof. In any of the methods of the first to tenth aspects, a compound of the disclosure is EVE18-(G⁷ to K), or a pharmaceutically acceptable form thereof. In any of the methods of the first to tenth aspects, a compound of the disclosure is EVE18 (G⁷ to H), or a pharmaceutically acceptable form thereof. In any of the methods of the first to tenth aspects, a compound of the disclosure is EVE18 (G⁷ to E), or a pharmaceutically acceptable form thereof. In any of the methods of the first to tenth aspects, a compound of the disclosure is EVE18 (G⁷ to A), or a pharmaceutically acceptable form thereof. In any of the following methods of the first to tenth aspects, the compound can be either EVE14 or EVEmR14L with equivalence. In any of the methods of the first to tenth aspects, a compound of the disclosure is EVE14 (or EVEmR14L, for example), or a pharmaceutically acceptable form thereof. In any of the methods of the first to tenth aspects, a compound of the disclosure is EVE14 (G⁵ to K), or a pharmaceutically acceptable form thereof. In any of the methods of the first to tenth aspects, a compound of the disclosure is EVE14 (G⁵ to R), or a pharmaceutically acceptable form thereof. In any of the methods of the first to tenth aspects, a compound of the disclosure is EVE14 (G⁵ to H), or a pharmaceutically acceptable form thereof. In any of the methods of the first to tenth aspects, a compound of the disclosure is EVE14 (G⁵ to E), or a pharmaceutically acceptable form thereof. In any of the methods of the first to tenth aspects, a compound of the disclosure is EVE14 (G⁵ to A), or a pharmaceutically acceptable form thereof. In any of the methods of the first to tenth aspects, a compound of the disclosure is Rev-EVE14, or a pharmaceutically acceptable form thereof. In any of the methods of the first to tenth aspects, a compound of the disclosure is Rev-EVE14 (G¹⁰ to K), or a pharmaceutically acceptable form thereof. In any of the methods of the first to tenth aspects, a compound of the disclosure is Rev-EVE14 (G¹⁰ to R), or a pharmaceutically acceptable form thereof. In any of the methods of the first to tenth aspects, a compound of the disclosure is Rev-EVE14 (G¹⁰ to H), or a pharmaceutically acceptable form thereof. In any of the methods of the first to tenth aspects, a compound of the disclosure is Rev-EVE14 (G¹⁰ to E), or a pharmaceutically acceptable form thereof. In any of the methods of the first to tenth aspects, a compound of the disclosure is Rev-EVE14 (G¹⁰ to A), or a pharmaceutically acceptable form thereof. In any of the methods of the first to tenth aspects, a compound of the disclosure is 2F-EVE14, or a pharmaceutically acceptable form thereof. In any of the methods of the first to tenth aspects, a compound of the disclosure is 3F-EVE14, or a pharmaceutically acceptable form thereof. In any of the methods of the first to tenth aspects, a compound of the disclosure is 3F-EVE18, or a pharmaceutically acceptable form thereof. In any of the methods of the first to tenth aspects, a compound of the disclosure is 4F-EVE18, or a pharmaceutically acceptable form thereof. In any of the methods of the first to tenth aspects, a compound of the disclosure is 4F, 2L-EVE18, or a pharmaceutically acceptable form thereof. In any of the methods of the first to tenth aspects, a compound of the disclosure is Rev-4F, 2L-EVE18, or a pharmaceutically acceptable form thereof. In any of the methods of the first to tenth aspects, a compound of the disclosure is 3F, 2L-EVE14, or a pharmaceutically acceptable form thereof. In any of the methods of the first to tenth aspects, a compound of the disclosure is Rev-3F, 2L-EVE14, or a pharmaceutically acceptable form thereof. In any of the methods of the first to tenth aspects, a compound of the disclosure is 4F, or a pharmaceutically acceptable form thereof. In any of the methods of the first to tenth aspects, a compound of the disclosure is Rev-4F, or a pharmaceutically acceptable form thereof.

In any of the methods of the first to tenth aspects, a compound of the disclosure enumerated above is complexed with natural or artificial lipids including but not limited to phospholipids and detergents.

In any of the methods of the first to tenth aspects, a compound of the disclosure is in the form of a pharmaceutically acceptable salt, solvate, or hydrate. A compound of the disclosure may be formulated as a pharmaceutically acceptable salt, e.g., acid addition salt, and complexes thereof. The preparation of such salts can facilitate the pharmacological use by altering the physical characteristics of the agent without preventing its physiological effect. Examples of useful alterations in physical properties include, but are not limited to, increasing the solubility to facilitate administering higher concentrations of the drug.

In any of the methods of the first to tenth aspects, a compound, a pharmaceutically acceptable form thereof or a mixture of the disclosure is prepared in a manner to stabilize it from low or high acid solutions; such a preparation may be but is not limited to enteric coating.

In any of the methods of the first to tenth aspects, a compound of the disclosure is a cationic or zwitterionic amphipathic peptide helix, or a pharmaceutically acceptable form thereof.

In any of the methods of the first to tenth aspects, a compound of the disclosure is a viral membrane envelope-active lipid, [lipid-peptide] composite, a [lipid-charged/hydrophobic compound] composite or a pharmaceutically acceptable form thereof.

In any of the methods of the first to tenth aspects, a compound of the disclosure is a cationic orv zwitterionic amphipathic peptide, or a pharmaceutically acceptable form thereof comprised of L-amino acid enantiomers.

In any of the methods of the first to tenth aspects, a compound of the disclosure is a cationic or zwitterionic amphipathic peptide, or a pharmaceutically acceptable form thereof comprised of D-amino acid enantiomers.

In any of the methods of the first to tenth aspects, a compound of the disclosure is a cationic or zwitterionic amphipathic peptide, or a pharmaceutically acceptable form thereof comprised of both D- and L-amino acid enantiomers.

In any of the methods of the first to tenth aspects, a compound of the disclosure is a cationic or zwitterionic molecule, or a pharmaceutically acceptable form thereof whose hydrophobic and hydrophilic faces. ionic composition and surface charge distribution mimic those of the peptides enumerated here.

### Dosage

A variety of doses of a compound of the disclosure may be used with the methods and in the compositions described herein. The dosage of the compound of the disclosure will vary depending on the condition being treated and the state of the subject. Suitable dose ranges are from 0.001 to 100 mg/kg, such as but not limited to, 0.001 to 25 mg/kg, 0.001 to 15 mg/kg, 0.001 to 10 mg/kg, 0.001 to 8 mg/kg, 0.001 to 6 mg/kg, 0.001 to 5 mg/kg, 0.001 to 4 mg/kg, 0.001 to 3 mg/kg, 0.001 to 2 mg/kg, or 0.001 to 1 mg/kg. Suitably, the dose contains 0.005 to 5 mg/kg of a compound of the disclosure, such as, but not limited to, 0.005 to 3 mg/kg, 0.005 to 2 mg/kg, 0.005 to 1 mg/kg, or 1 to 2 mg/kg.

### Compositions

Accordingly, the present invention includes a pharmaceutical composition for use in the methods described herein, comprising a compound of the disclosure or pharmaceutically acceptable form thereof, together with a pharmaceutically acceptable excipient suitable for administration to a subject. Such pharmaceutical compositions may further comprise one or more additional active agents. In certain embodiments, the administration route is parenteral, pulmonary, intra-nasal, buccal, oral-gastric or intravenous administration. Such pharmaceutical compositions may be used in the manufacture of a medicament for use in the methods of treatment and prevention described herein. The compounds of the disclosure are useful in both free form and in the form of pharmaceutically acceptable salts.

Pharmaceutically acceptable excipients are also well-known to those who are skilled in the art. The choice of excipient will be determined in part by the particular compound(s), as well as by the particular method used to administer the composition. Accordingly, there is a wide variety of suitable formulations of the pharmaceutical composition of the present invention. The following methods and excipients are merely exemplary and are in no way limiting. Suitable excipients include solvents such as water, alcohol, and propylene glycol, solid absorbants and diluents, surface active agents, suspending agent, tableting binders, lubricants, flavors, enteric coatings, and coloring agents. The pharmaceutically acceptable excipients can include polymers and polymer matrices. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the pharmaceutically acceptable excipient is chemically inert to the active agents in the composition and has no detrimental side effects or toxicity under the conditions of use. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

In addition to inert excipients, other components of a pharmaceutical composition may include food or food-like materials which when combined with a compound of this disclosure or a crude or purified [peptide/charged molecule-enveloped virus] admixture produce a protective effect on human or non-human subjects against exposure to virus or an ameliorative effect following their exposure or infection with a virus.

Surfactants such as, for example, detergents, are also suitable for use in the formulations. Specific examples of surfactants include but are not limited to polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺ R′R″R″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

The compounds of the present disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the present disclosure are administered in therapeutically effective amount, whether alone or as a part of a pharmaceutical composition. The therapeutically effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

In these pharmaceutical compositions, the compound(s) of the present disclosure will ordinarily be present in an amount of about 0.5-95% weight based on the total weight of the composition. Multiple dosage forms may be administered as part of a single treatment.

The active agent can be administered orally or enterally in solid dosage forms, such as capsules, tablets, powders, food pellets or food-like mixtures, or in liquid dosage forms, such as milk, elixirs, syrups, suspensions or a liquid spray. It can also be administered parenterally, in sterile liquid dosage forms. The compound(s) of the present disclosure can also be administered intranasally (nose drops) or by inhalation via the pulmonary system, such as by propellant based metered dose inhalers or dry powders inhalation devices. Other dosage forms include topical administration, such as administration transdermally, via patch mechanism or ointment. Other dosage forms include solutions or mixture given orally. Oral solutions may include but are not limited to sprays or admixtures containing food or food-like material for the target species.

Formulations suitable for enteral or oral administration may be liquid solutions, such as a therapeutically effective amount of the compound(s) dissolved in diluents, such as milk, water, saline, buffered solutions, infant formula, other suitable excipients, or combinations thereof. The compound(s) can then be mixed to the diluent just prior to administration. In an alternate embodiment, formulations suitable for enteral or oral administration may be capsules, sachets, tablets, lozenges, troches, food or food-like mixtures. In each embodiment, the formulation may contain a predetermined amount of the compound(s) of the present disclosure, as solids or granules, powders, suspensions and suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients.

Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) can be administered in a physiologically acceptable diluent in a pharmaceutically acceptable excipient, such as a sterile liquid or mixture
of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants including phospholipids.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

In certain embodiments, the compound of the disclosure is delivered as an aerosol comprising a plurality of solid particles. Such aerosols may be produced by any means known in the art, including but not limited to, an insufflator and a metered dose inhaler. Suitable formulations
for administration by insufflation include finely comminuted powders. In one embodiment, the compound of the disclosure is present as a powder and contained in a cartridge which is pierced or otherwise opened to allow the powder to be drawn through the device when a subject inhales or on the activation of a pump. Suitable excipients include, but are not limited to diluents and surfactants as is known in the art. Metered dose inhalers are pressurized aerosol dispensers, typically containing a suspension or solution of the compound of the disclosure in a liquid propellant. Suitable propellants include certain chlorofluorocarbon compounds, for example, dichlorodifluoromethane, trichlorofluoromethane, dichlorotetrafluoroethan-e and mixtures thereof.

The compound(s) of the present disclosure can be formulated into aerosol formulations to be administered via nasal or pulmonary inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

For nasal or pulmonary delivery, a composition comprising a therapeutically effective amount of a compound of the disclosure is administered to the subject via inhalation into the nose/respiratory system. Such a composition may comprise a particulate form of the compound of the disclosure. The particulate forms of the compound of the disclosure (whether solid or liquid) are produced to be of a size suitable for inhalation therapy such that, for example, the particulate forms of the compound of the disclosure pass through the mouth upon inhalation and into the bronchi and alveoli of the lungs. In one embodiment, a suitable size range for such particulates is in the range of 0.5 to 15 microns. The compound of the disclosure administered by inhalation may be in the form of a dry powder, a mist, or an aerosol.

In certain embodiments, the compound of the disclosure is delivered as an aerosol comprising a plurality of liquid particles. Such aerosols may be produced by any means known in the art, including but not limited to, a nebulizer, a pressure driven nebulizer, and an ultrasonic nebulizer.

The compound(s) of the present disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the present invention typically comprise the drug or drug formulation to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 7.0. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonaryly respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

To formulate compositions for oral-gastric delivery, the active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. The agent may be combined with food or food-like material palatable to the target species to create an oral-gastric formulation that is readily ingested by human or non-human subjects in a controlled setting or in natural settings. When the composition for oral-gastric delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of about ⅓ to 3, more typically ½ to 2, and most often ¾ to 1.7. The compound(s) of the present disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application. The use of a selected carrier in this context may result in promotion of absorption of the active agent.

The compounds of the present disclosure may alternatively contain as pharmaceutically acceptable excipient substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, and the like.

### EXAMPLES

### Example 1: Preparation of Peptides

Peptides were synthesized using solid phase peptide synthesis method using MBHA resin. The peptide resin was acetylated and peptides were cleaved from the resin using TFA (in presence of scavengers to protect Trp) to yield the stable peptides. Peptides were purified using preparative HPLC and purity was confirmed by analytical HPLC and mass spectrometry. For EVE5, Fmoc-aminohexanoic acid (Aha) was added to the amino terminus of the arginine-rich region. After removal of Fmoc-group, the peptide resin was acetylated and peptide further processed as described above. Any of the peptides disclosed herein may be used in the form of a peptide-lipid complex. The peptides disclosed easily associates with a variety of lipids, such as, but not limited to, dimyristoylphosphatidylcholine (DMPC), egg PC, palmitoyloleylphosphatidyl choline (POPC). In one embodiment, the lipid is DMPC. Peptide and lipid mixtures (1:1 to 1:20 by weight ratio) are prepared by spontaneous mixing. The peptide:lipid complex is sterilized by membrane filtration.

### Example 2: EVE5 Exhibits Potent Anti-Viral Activity Against SARS-COV-2 Alpha

EVE5 was tested for antiviral activity against an exemplary pathogenic enveloped virus (SARS-COV-2 Alpha). In this example, Vero-E6 cells were grown in complete EMEM containing 10% FBS and Pen/Strep at 37° C. and 5% CO2. Once cells reached desired confluence (for example, 80%). SARS-COV-2 Alpha virus was pre-incubated with varying concentrations of EVE5 for 60 minutes in ˜20 microliters of complete EMEM. The EVE5-treated SARS-COV2 Alpha virus was then added to fresh cell medium at a 1:10 dilution. Cell medium was aspirated from Vero-E6 cells and replaced with the complete medium containing [peptide-virus]. Cells were incubated for 24 hours at 37° C. with 5% CO2. After incubation, the cells were analyzed for SARS-COV-2 spike protein by immunohistochemistry.

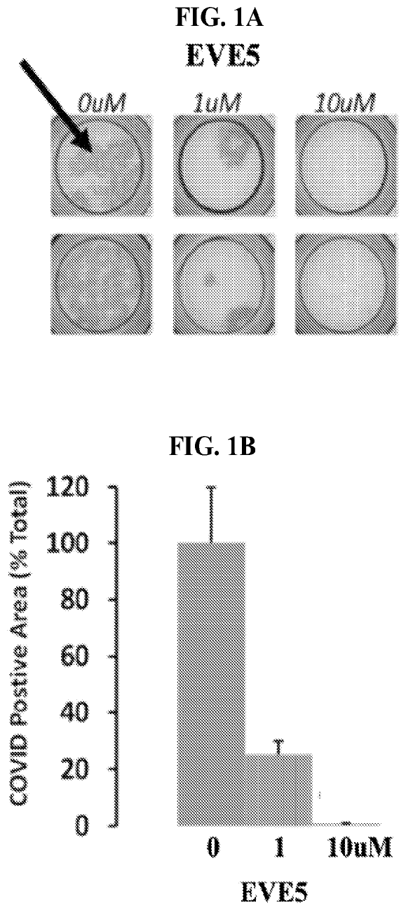

In FIG. 1A, the circle in each panel encloses a region that is confluent with Vero cells. The cells were treated with SARS-COV2-Alpha pre-incubated with 0 to 10 μM of EVE5. After incubation for 24 hr the enclosed regions were analyzed for their content of SARS-COV-2 spike protein by immunohistochemistry. In FIG. 1A, the darkened area in each circle shows immuno-reaction against the SARS-COV-2 spike protein, with a reduction in SARS-COV-2 spike protein immunoreactivity (SARS-COV-2 positive area) indicating EVE5 is effective in inhibiting SARS-CoV-2 replication. FIG. 1B quantifies the data in FIG. 1A. Results are the mean±SEM.

FIGS. 1A and B show treatment with EVE5 decreased SARS-COV-2 infectivity. At a concentration of luM, EVE5 reduced the SARS-COV-2 positive area by approximately 80%. At a concentration of 10 μM, EVE5 reduced the SARS-COV-2 positive area to essentially 0.

### Example 3: EVEmR18L Shows Antiviral Activity Against SARS-COV-2 Alpha in Vero-E6 Cells

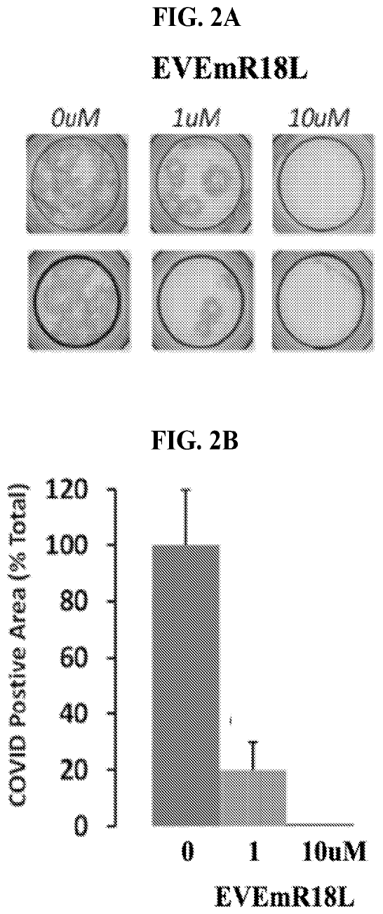

In this example, EVEmR18L was tested for antiviral activity against an exemplary pathogenic enveloped virus (SARS-COV-2 Alpha). The methodology used is that described in Example 2. The results are shown in FIGS. 2A and B. Results are presented as the mean±SEM.

Treatment with EVEmR18L decreased SARS-COV-2 infectivity. At a concentration of 1 μM, EVEmR18L reduced the SARS-COV-2 positive area by approximately 80%. At a concentration of 10 μM, EVEmR18L reduced the SARS-COV-2 positive area to essentially 0.

### Example 4: EVE4 Exhibits Antiviral Activity Against SARS-COV-2 Alpha in Vero-E6 Cells

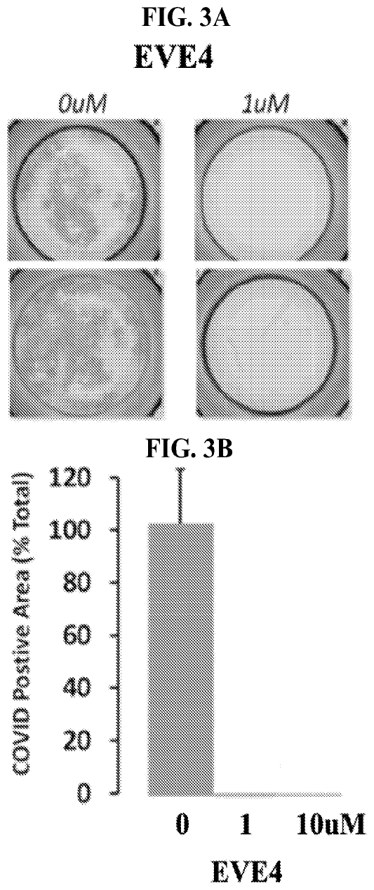

In this example, EVE4 was tested for antiviral activity against an exemplary pathogenic enveloped virus (SARS-COV-2 Alpha). The methodology used is that described in Example 2. The results are shown in FIGS. 3A and B. Results are presented as the mean±SEM.

Treatment with EVE4 significantly decreased SARS-COV-2 infectivity. At a concentration of 1 μM, EVE4 reduced the SARS-COV-2 positive area to essentially 0.

### Example 5: Antiviral Activity of EVE4, EVE5, and EVEmR18L in Comparison to Remdesivir

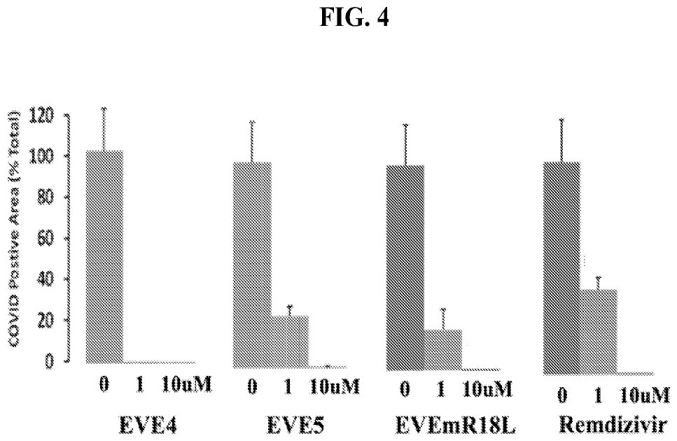

In this example, the antiviral activity of remdesivir was compared to EVE4, EVE5, and EVEmR18L. Remdesivir is an investigational nucleotide analog with broad-spectrum antiviral activity. It is currently being tested in clinical trials for the treatment of COVID-19. The methodology for determination of remdesivir antiviral activity is as described in Example 2. The results are shown in FIG. 4 (with the data from FIGS. 1B, 2B, and 3B for EVE5, EVEmR18L, and EVE4, respectively, shown for comparison). Results are presented as the mean±SEM.

Treatment with remdesivir, EVE4, EVE5, and EVEmR18L decreased SARS-COV-2 infectivity. EVE4, EVE5, and EVEmR18L displayed greater antiviral activity than remdesivir. At a concentration of luM, remdesivir reduced the SARS-COV-2 positive area approximately 65%. In comparison, at luM, EVE4 reduced the SARS-COV-2 positive area to essentially 0 and EVE5 and EVEmR18L reduced the SARS-COV-2 positive area by approximately 80%.

### Example 6: Antiviral Activity of EVE4, EVE5, and EVEmR18L

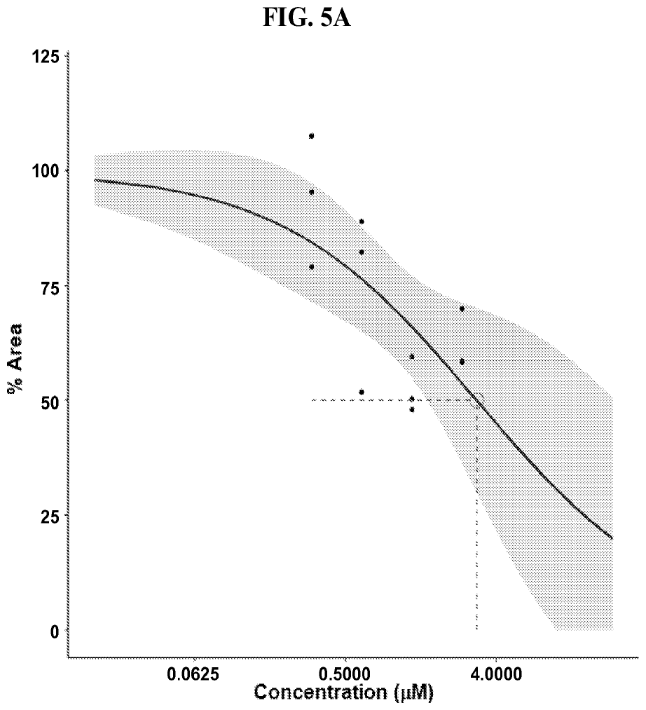

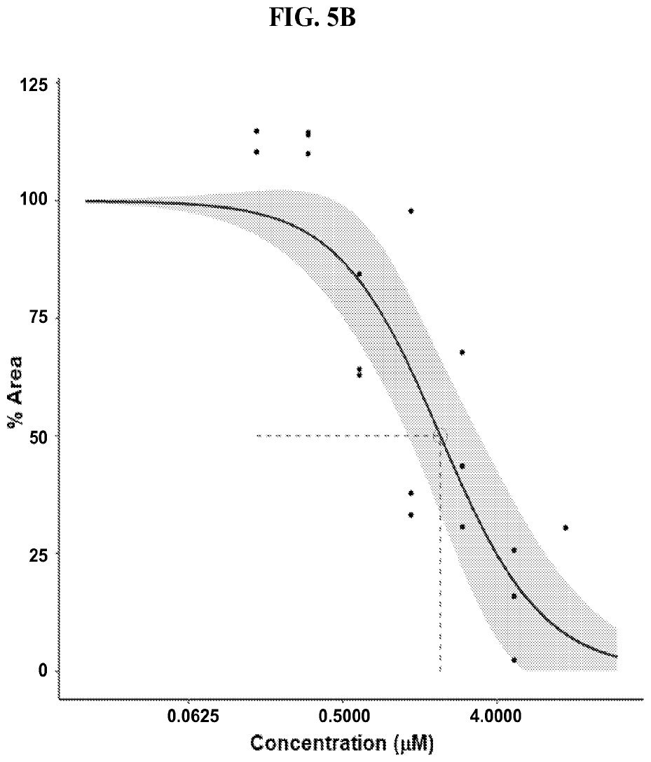

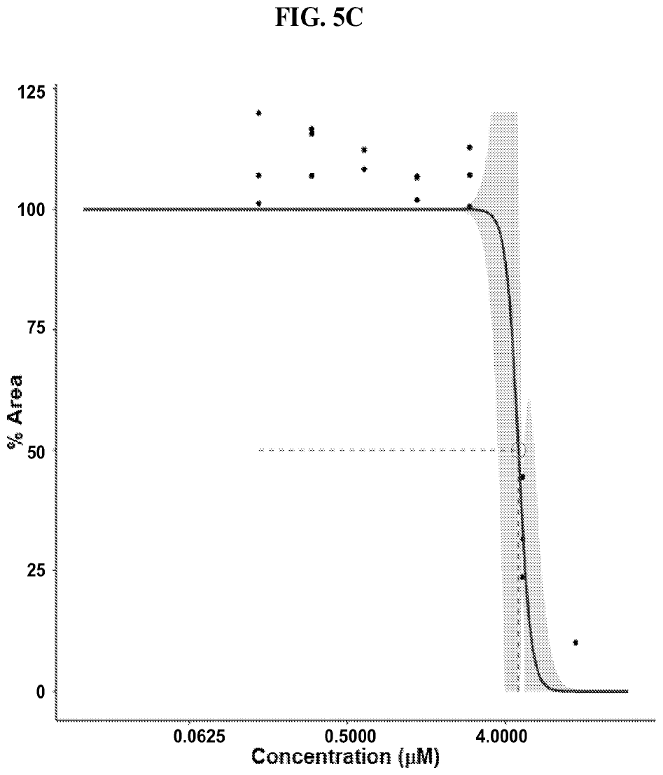

In this example, EVE4, EVE5, and EVEmR18L were tested for antiviral activity against an exemplary pathogenic enveloped virus (SARS-COV-2 Alpha) as described in Example 2 using a range of concentrations to determine the IC50 values for each peptide. The results are shown in FIGS. 5A to 5C for EVE4, EVE5, and EVEmR18L, respectively.

Consistent with the results of Examples 2 to 5, EVE4, EVE5 and EVEmR18L significantly decreased SARS-COV-2 infectivity. IC50 values were between 3 and 4.5 μM for these peptides.

Examples 2-6 show that the compounds of the disclosure are in vitro antiviral agents and validate their use as therapeutics for the treatment and prevention of viral disease in subjects.

### Example 7: EVE4, EVE5, and EVEmR18L are not Cytotoxic

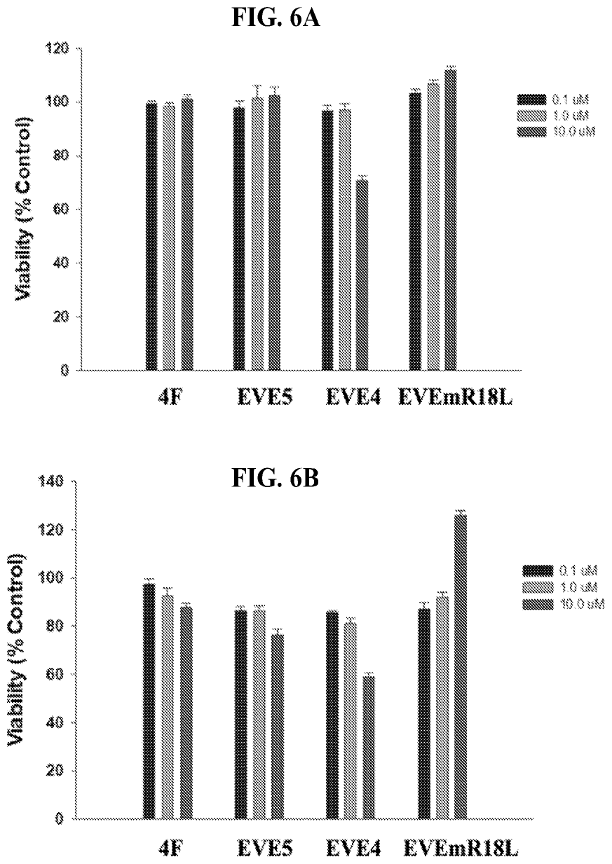

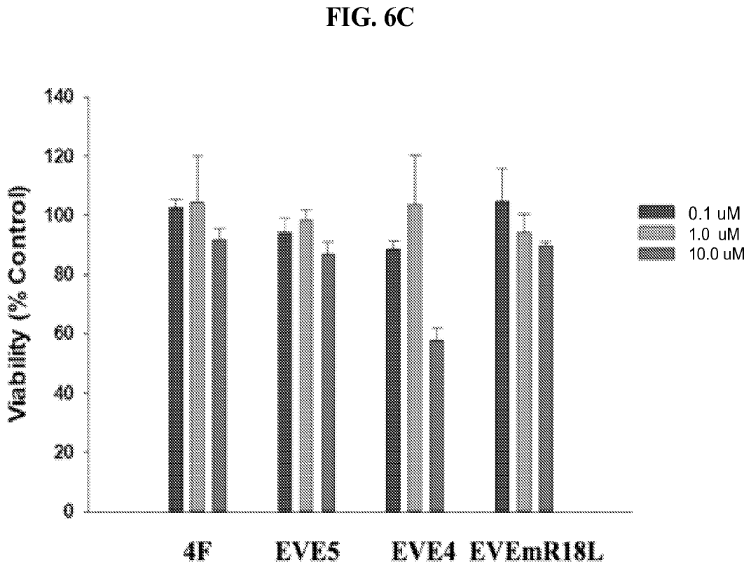

The peptides of the disclosure did not exhibit cytotoxic effects in Vero-E6 cells. Data for peptide 4F (Ac-DWFKAFYDKVAEKFKEAF-NH₂; SEQ ID NO: 34) is also provided. In this example, Vero-E6 cells (96 well plate) were grown in EMEM containing 10% FBS and Pen/Strep at 37° C. and 5% CO₂. Once cells reached 80% confluence, medium was replaced with fresh complete medium containing the indicated concentration of peptide (FIGS. 6A-B) or peptide complexed with DMPC (1:5 ratio weight:weight). Incubation period proceeded for 24 or 48 hrs. Cells were washed and replaced with complete medium containing fluorescent Reliablue reagent. Viability was then measured. Data are presented as percent viability compared to control (no additions). Results are presented as the mean±SEM.

FIGS. 6A-C show the peptides of the disclosure do not display significant cytotoxic activity in Vero-E6 cells.

### Example 8: Pre-Treating H1N1A-California with EVE14 Suppresses Weight Loss in Female Mice Challenged with these Viral Preparations

In this example we assessed the effect of pre-incubating H1N1A with the EVE14 peptide on H1N1 pathogenicity. Three sets of mice were used. H1N1A-California virus (1200 pfu) was incubated in 30 microliters of saline in the presence of 10 micromolar EVE14 for 1, 5, and 60 minutes. These three [peptide-H1N1] preparations were given to mice intranasally. Mice were returned to their cages and their bodyweight was measured daily for 14 days.

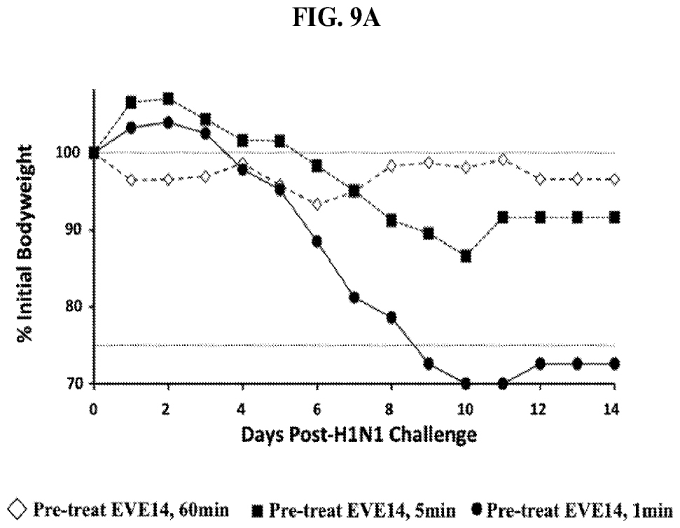

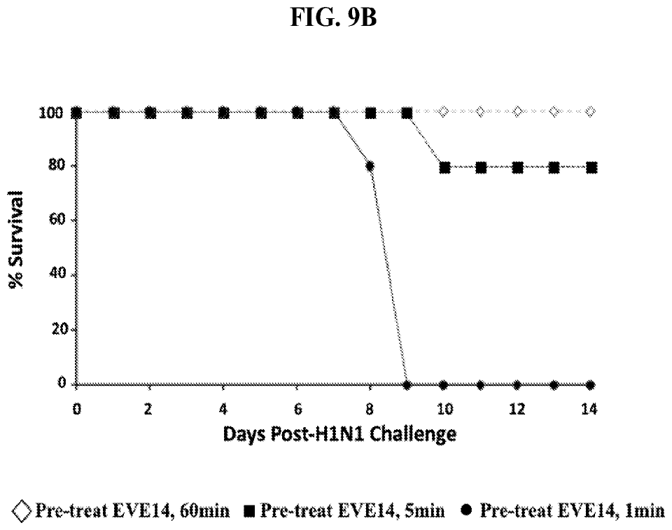

FIG. 9A demonstrates that EVE14 inactivates H1N1 in a time-dependent manner, rendering H1N1 incapable of inducing weight loss in female C57 mice.

### Example 9: Pre-Treating H1N1A-California with EVE14 Prevents Mortality in Female C57 Mice Challenged with these Viral Preparations

We assessed the effect of pre-incubating H1N1A with the EVE14 peptide on H1N1 pathogenicity. Three sets of mice were used. H1N1A-California virus (1200 pfu) was incubated in 30 microliters of saline in the presence of 10 micromolar EVE14 for 1, 5, and 60 minutes. These three [peptide-H1N1] preparations were given to female C57 mice intranasally. Mice were returned to their cages and frank death or bodyweight loss was measured daily for 14 days. A 25% decrease in bodyweight was considered equivalent to death.

FIG. 9B demonstrates that EVE14 inactivates H1N1 in a time-dependent manner, rendering H1N1 incapable of inducing death in female C57 mice.

### Example 10: Administering H1N1 Inactivated by Pre-Incubation with EVE14 to Female C57-B⅙J Mice Produces Adaptive Immunity Against Active H1N1

All female mice challenged with H1N1A that had been pre-incubated with EVE14 for 60 minutes survived this initial challenge and lost little weight. Several weeks later these mice were challenged a second time with 1200 pfu of active H1N1. Mice were returned to their cages and their bodyweight measured daily for 14 days.

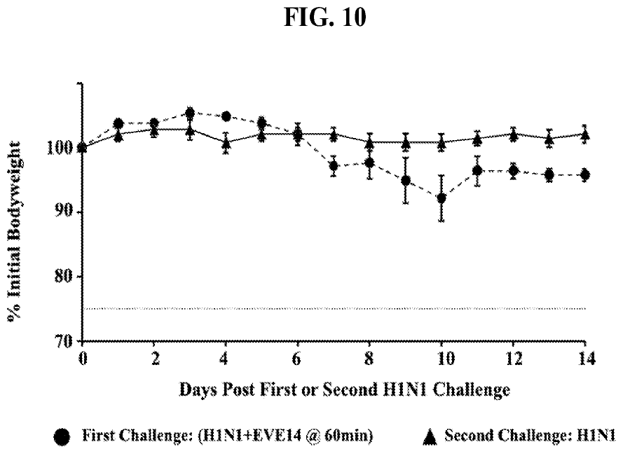

FIG. 10 shows that no mouse in the group which underwent a second H1N1 challenge lost significant bodyweight. That is, EVE14 inactivates HINI to protect against an initial viral challenge and this treatment protects against a subsequent challenge with active virus. This result is distinct from the case in which a mouse may survive an initial exposure to H1N1. In this case, significant loss of bodyweight occurs and mice exhibit pathological symptoms. In contrast, mice given EVE14-treated H1N1 exhibited no bodyweight loss or other sign of pathology but they still were immune to the active H1N1 given in the second challenge.

### Example 11: Sub-Cutaneous EVE14 Reduces Weight Loss in Female C57 Mice that were Given Intranasal H1N1

In this example, we assessed whether EVE14 given sub-sutaneously to female C57 mice following intranasal H1N1 challenge could reduce the bodyweight loss seen in such animals.

One group of female C57-BL/6J mice were administered 1200 pfu H1N1 and returned to their cages. A second group of mice were given 300 micrograms of EVE14 in saline sub-cutaneously 2 hr prior to the intranasal administration of 1200 pfu H1N1. These mice also received sub-cutaneous EVE14 boosters of 150 micrograms at 24 hr and 48 hr following H1N1. A third group of mice received 300 micrograms of EVE14 in saline sub-cutaneously 24 hr prior to the intranasal administration of 1200 pfu of H1N1. These mice received 150 micrograms of EVE14 sub-cutaneously at 24 hr, 48 hr, 72 hr and 96 hr after H1N1. Bodyweight of all groups of mice were measured daily for 14 days.

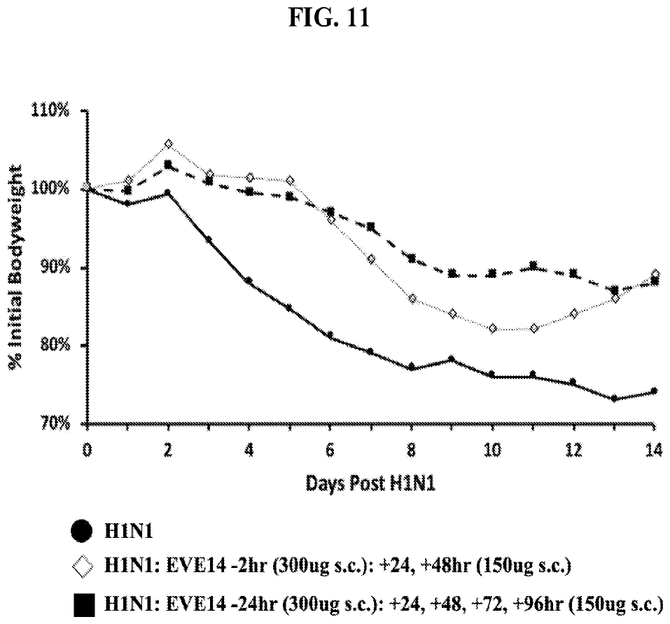

FIG. 11 shows female C57 mice given H1N1 alone lost significant bodyweight in the seven days following intranasal administration of the active virus. In contrast, mice given EVE14 sub-cutaneously before and after H1N1 had a significant delay in their weight loss which also was blunted compared to untreated mice.

### Example 12: Sub-Cutaneous EVE14 Improves Survival in Female C57 Mice Challenged with H1N1

Survival or bodyweight loss of less than 25% was measured in the three groups of mice studied in Example 11.

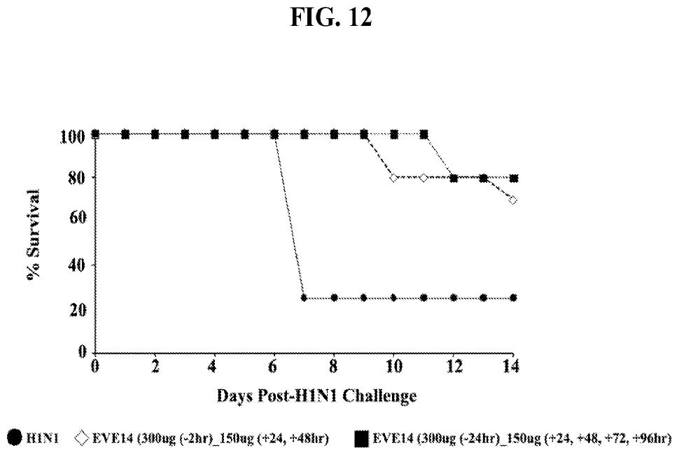

FIG. 12 shows that both EVE14 treatment groups exhibited significantly increased survival compared to the group of mice that received H1N1 alone.

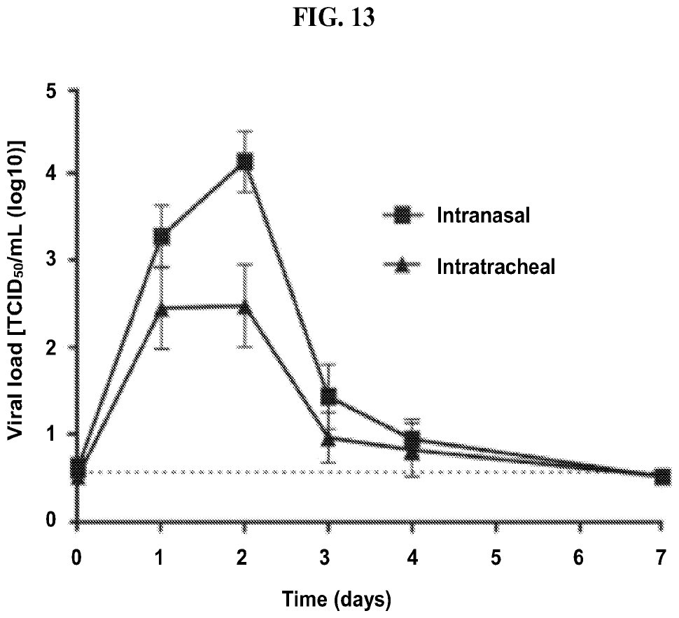

FIG. 13 show others' data that mouse viral loads peak 24-48 hrs after receiving intranasal or intratracheal H1N1. This data indicate that higher doses of EVE14 may be required to fully nullify the adverse effects of H1N1 if peptide is given 24 or more hours after infection.

### Example 13: Sub-Cutaneous EVE14 Produces Adaptive Immunity Against H1N1

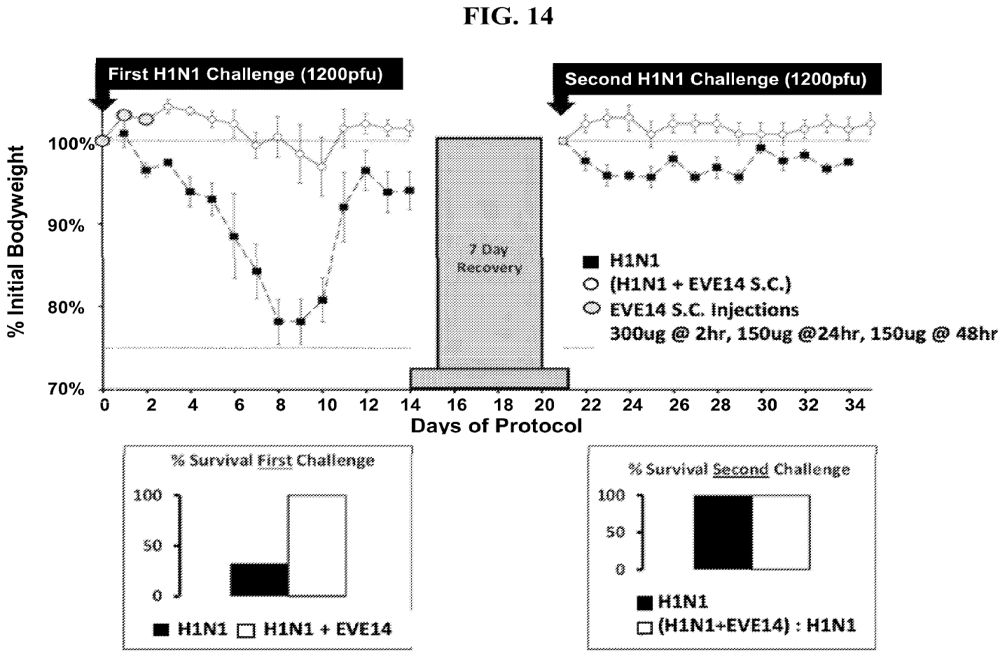

Typically, an 80% mortality occurs in female C57 mice challenged with H1N1 (FIG. 14 Lower left panel, solid bar). Treating naïve female C57BL/6J mice with 1200 pfu H1N1 causes significant bodyweight loss in the surviving mice, a loss from which they can recover (FIG. 14 Upper left panel, full squares). In mice that survive H1N1 challenge and recover from infection, a second challenge with H1N1-California does not affect mouse physiology (FIG. 14 Right panel, full squares). This is a manifestation of an adaptive immune response. That is, mice that survive an initial exposure to an enveloped virus do not respond to a second exposure.

Female C57BL/6J mice challenged intranasally with 1200 pfu H1N1 and given EVE14 sub-cutaneously exhibit a much reduced weight loss (FIG. 14 Left panel, open circles). A second challenge of these mice with 1200 pfu H1N1 given three weeks after the first does not elicit mortality or weight loss (FIG. 14 Upper and lower right panels open icons).

Sub-cutaneous EVE14 peptide thus suppresses the initial pathological response to H1N1 and protects mice from a second challenge 21 days later.

### Example 14: EVE14 Inactivates H1N1 so that it Cannot Cause Death in Female C57 Mice

One set of female C57B1-6J mice were given one intranasal dose of H1N1A-California (1200 pfu). They were returned to their cages and their survival was monitored for 14 days. Death was assessed as frank mortality or as a weight loss of 25% initial bodyweight. Six days following H1N1 dosing approximately 80% of these mice had died.

H1N1A-California (1200 pfu) was pre-incubated for ˜45 min with approximately 10 μM EVE14. Following this preincubation, EVE14-treated H1N1 were administered intranasally to female C57B1-6J mice. Mouse mortality was monitored for 14 days. No mice in this group died.

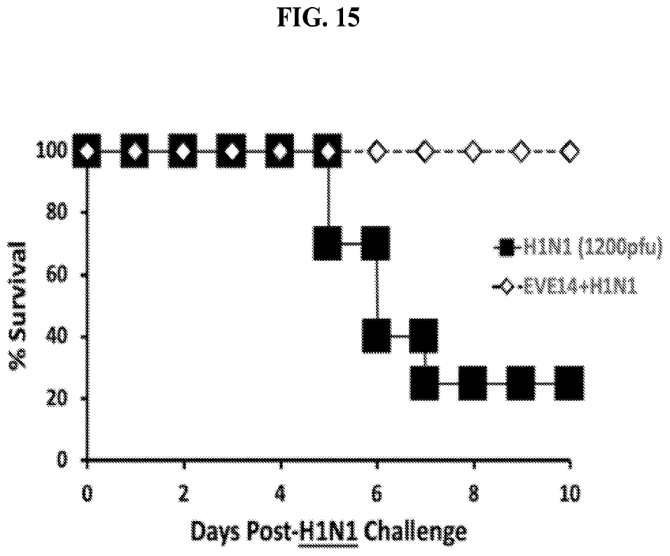

FIG. 15 demonstrates that EVE14 inactivates H1N1 and renders it incapable of inducing weight loss or frank death in female C57 mice.

### Example 15: EVE18 Inactivates H1N1 so that it Cannot Cause Death in Female C57 Mice

One set of female C57B1-6J mice were given one intranasal dose of H1N1A (1200 pfu). They were returned to their cages and their survival was monitored for 14 days. Death was assessed as frank mortality or as a weight loss of 25% initial bodyweight. Six days following H1N1 dosing approximately 80% of these mice had died.

H1N1A-California (1200 pfu) was pre-incubated for ˜45 min with approximately 10 μM EVE18. These EVE18-treated H1N1 were administered intranasally to female C57B1-6J mice. Mortality was monitored for 14 days. No mouse in this group died or lost significant bodyweight.

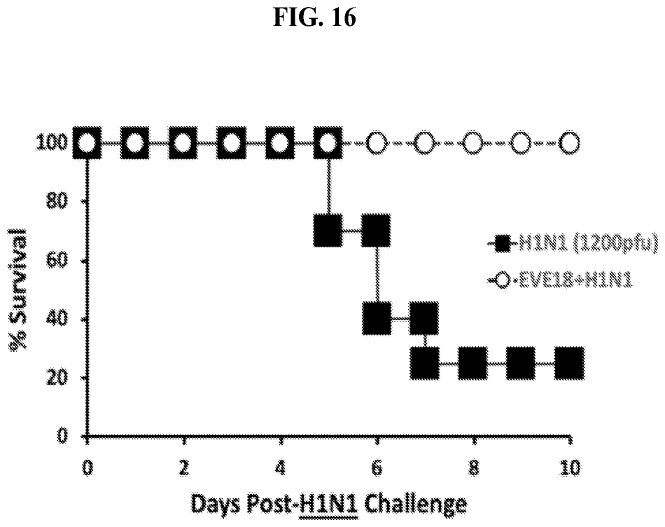

FIG. 16 demonstrates EVE18 inactivates H1N1 and renders it incapable of inducing weight loss or frank death in female C57 mice.

### Example 16: H1N1 Inactivated with EVE18 and Given Intranasally to Female C57B1-6J Mice Protects them from a Subsequent Challenge with Active H1N1

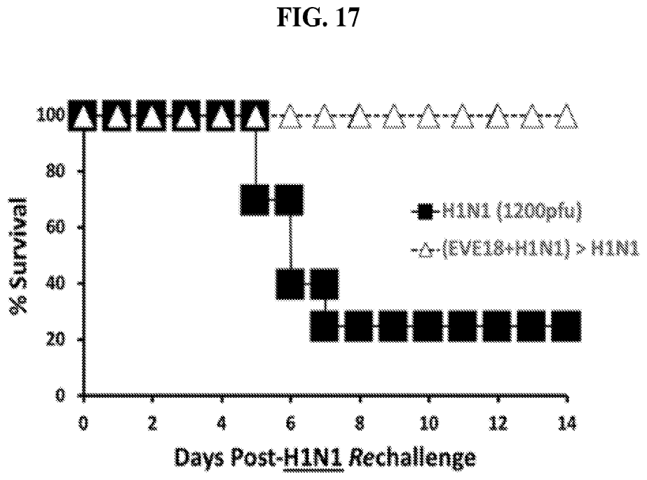

One set of female C57B1-6J mice were given one intranasal dose of H1N1A-California (1200 pfu). They were returned to their cages and their survival was monitored for 14 days. Death was assessed as frank mortality or as a weight loss of 25% initial bodyweight. Six days following H1N1 dosing approximately 80% of these mice died (FIG. 17, Closed squares).

H1N1A-California (1200 pfu) was pre-incubated for ˜45 min with approximately 10 μM EVE18. These EVE18-treated H1N1 were administered intranasally to female C57B1-6J mice. No mouse in this group died or lost significant bodyweight (FIG. 16, Open circles).

These mice were then REchallenged several weeks later with an intranasal dose of active H1N1A-California (1200 pfu). Mouse mortality was monitored for 14 days. No mouse in this group died during this period (FIG. 17, Open triangles).

FIG. 17 demonstrates that administering H1N1 that was treated with EVE18 to female C57B1-6J mice protects them from a subsequent challenge with active H1N1A. This non-pathogenic [EVE18-H1N1] formulation invokes a protective response against H1N1.

### Example 17: Remdizivir Offers Little Protection from SARS-COV-2 Alpha in Female hACE2 Knock-In Mice

Remdizivir is a viral RNA polymerase inhibitor and an anti-COVID therapeutic. We tested its efficacy in curbing mortality in female hACE2 Knock-in mice challenged with SARS-COV-2 Alpha.

SARS-COV-2 Alpha virus (54000 pfu) were administered intranasally to female hACE2 Knock-in mice. Mouse mortality was monitored for 10 days. Intranasal SARS-COV-2 Alpha caused ˜80% mortality in hACE2 during six days after dosing.

Female hACE2 Knock-in mice were treated with 25 mg remdizivir/kg bodyweight. This dose and timing are efficacious in lowering the progression of SARS-COV-2 in experimental animals. In our mice, this dose caused a small right shift in the Kaplan-Meier curve and an increase in mouse survival from 20% in untreated mice to 40% in remdizivir-treated animals.

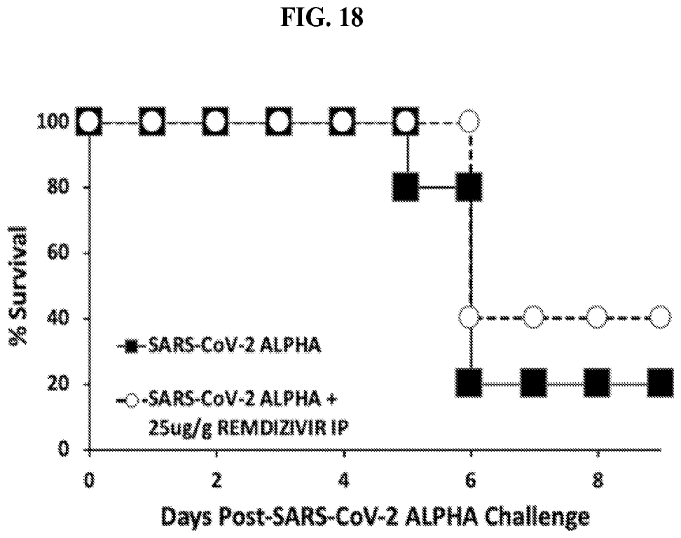

FIG. 18 shows only modest effects of a single dose of remdizivir are observed in hACE2 mouse mortality.

### Example 18: EVE14 Inactivates SARS-COV-2 Alpha Rendering it Incapable of Causing Death in Female hACE2 Knock-In Mice

One set of female hACE2 Knock-In mice was given one intranasal dose of active SARS-CoV-2 Alpha (54000 pfu). These mice were returned to their cages and their survival was monitored for 14 days as frank mortality. Between six and eight days after SARS-COV-2 Alpha dosing approximately 90% of these mice died.

SARS-COV-2 Alpha (54000 pfu) was pre-incubated for ˜45 min with 10 μM EVE14. EVE14-treated SARS-COV-2 Alpha then was administered intranasally to female hACE2 Knock-in mice. Mortality was monitored for 14 days. Ninety per-cent of these mice survived.

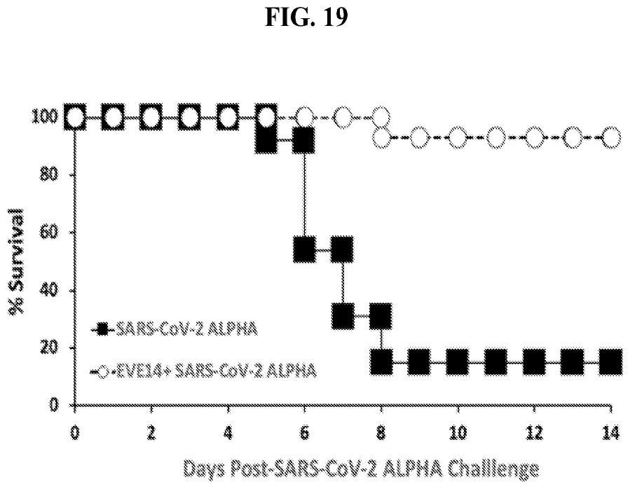

FIG. 19 demonstrates that EVE14 inactivates SARS-COV-2 Alpha and renders it incapable of causing death in female hACE2 Knock-in mice.

### Example 19: EVE18 Inactivates SARS-COV-2 Alpha Rendering it Incapable of Causing Death in Female hACE2 Knock-In Mice

One set of female hACE2 Knock-in mice were given one intranasal dose of active SARS-CoV-2 Alpha (54000 pfu). These mice were returned to their cages and their survival was monitored for 14 days as frank mortality. Between six- and eight-days following SARS-COV-2 Alpha dosing only 80% of these mice died.

SARS-COV-2 Alpha (54000 pfu) was pre-incubated for ˜45 min with 10 μM EVE18. EVE18-treated SARS-COV-2 Alpha then was administered intranasally to female hACE2 Knock-in mice. Mouse mortality was monitored for 14 days. All mice in this group survived.

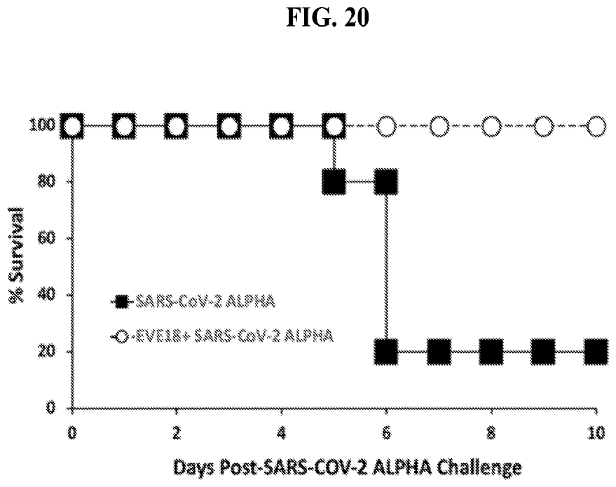

FIG. 20 demonstrates that EVE18 inactivates SARS-COV-2 Alpha and renders it incapable of causing death in female hACE2 Knock-in mice.

### Example 20: Challenging Female hACE2 Knock-In Mice with SARS-COV-2 Alpha Treated with EVE18 Protects them from Death Following a Subsequent Challenge with Active SARS-COV-2 Alpha. Virolytics Protect Against EVs in a Homologous Manner

One set of female hACE2 Knock-in mice were given one intranasal dose of active SARS-CoV-2 Alpha (54000 pfu). These mice were returned to their cages and their survival was monitored for 14 days as frank mortality. Between six- and eight-days following SARS-COV-2 Alpha dosing 80% of these mice died.

SARS-COV-2 Alpha (54000 pfu) was pre-incubated for ˜45 min with ˜10 μM EVE18. EVE18-treated SARS-COV-2 Alpha was administered intranasally to female hACE2 Knock-in mice. Mouse mortality was monitored for 14 days. All mice in this group survived.

Eight weeks after this initial challenge with EVE18 inactivated SARS-COV-2 Alpha, these female hACE2 Knock-in mice were Rechallenged with SARS-COV-2 Alpha (54000 pfu). Their mortality was monitored for 14 days. ˜ 75% of these rechallenged mice survived.

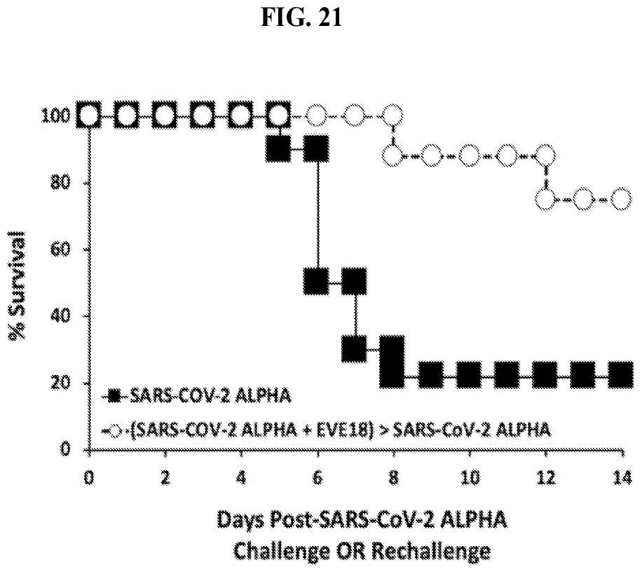

FIG. 21 demonstrates that challenging female hACE2 Knock-in mice with EVE18 inactivated SARS-COV-2 Alpha protects them from death as a result of a subsequent Rechallenge with active SARS-COV-2 Alpha.

### Example 21: EVE18 Inactivates SARS-COV-2 Delta Rendering it Incapable of Causing Death in Female hACE2 Knock-In Mice

One set of female hACE2 Knock-in mice were given one intranasal dose of active SARS-CoV-2 Delta (54000 pfu). These mice were returned to their cages and their survival was monitored for 14 days as frank mortality. Between eight- and ten-days following SARS-COV-2 Delta dosing 75% of these mice died.

SARS-COV-2 Delta (54000 pfu) was pre-incubated for ˜45 min with 10 μM EVE18. EVE18-treated SARS-COV-2 Delta was administered intranasally to female hACE2 Knock-in mice. Mouse mortality was monitored for 14 days. All mice in this group survived.

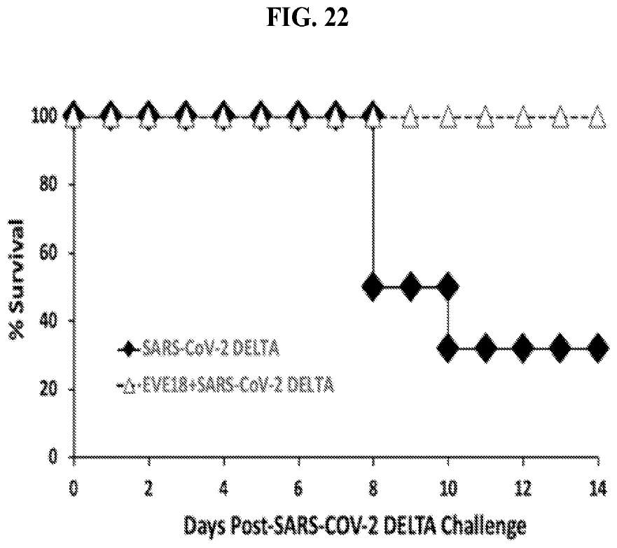

FIG. 22 demonstrates that EVE18 inactivates SARS-COV-2 Delta and renders it incapable of inducing death in hACE2 Knock-in mice.

### Example 22: Challenging Female hACE2 Knock-In Mice with SARS-COV-2 Alpha Treated with EVE18 Protects them from Death Following a Subsequent Challenge with Active SARS-COV-2 Delta. Virolytcs can Protect Against EVs in a Heterologous Manner

One set of female hACE2 Knock-in mice were given one intranasal dose of active SARS-CoV-2 Delta (54000 pfu). These mice were returned to their cages and their survival was monitored for 14 days as frank mortality. Between eight- and ten-days following SARS-COV-2 Delta dosing 75% of these mice died (FIG. 22).

SARS-COV-2 Alpha (54000 pfu) was pre-incubated for ˜45 min with 10 μM EVE18. EVE18-treated SARS-COV-2 Alpha was administered intranasally to two groups of female hACE2 Knock-in mice. Mortality was monitored for 14 days. All mice in this group survived (FIG. 20, Open circles, e.g.).

Nine and seven weeks after this initial challenge with EVE18 inactivated SARS-COV-2 Alpha, these two groups of female hACE2 Knock-in mice were Rechallenged with SARS-COV-2 Delta (54000 pfu). Their mortality was monitored for 14 days. 85% survival was recorded in these two group of animals.

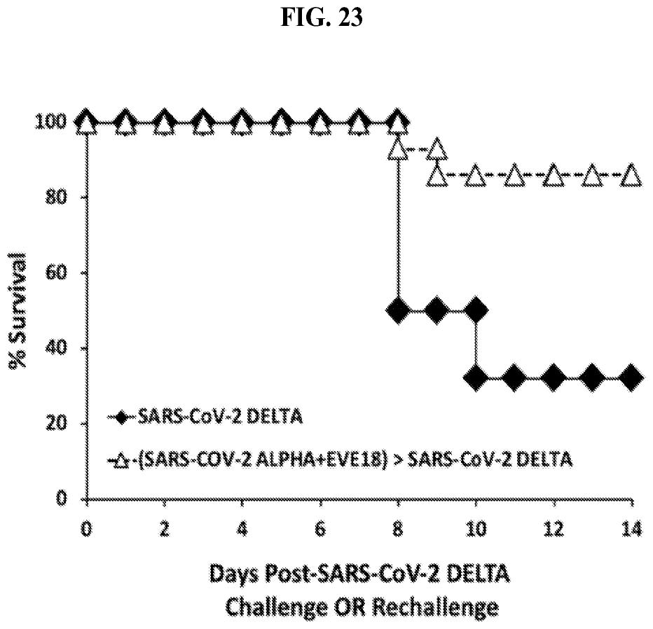

FIG. 23 demonstrates that challenging female hACE2 Knock-in mice with EVE18-inactivated SARS-COV-2 Alpha protects them from death by a subsequent Rechallenge with active SARS-COV-2 Delta virus. This protection persisted for an 11 week total experimental period. Thus, a SINGLE virolytic treatment protects against SARS-COV-2 mortality in a heterologous manner AND over an extended period of time.

### Example 23: EVE18 Decreases Mortality when Given Therapeutically to Female hACE2 Knock-In Mice Challenged with SARS-COV-2 Alpha

One set of female hACE2 Knock-in mice were given one intranasal dose of active SARS-CoV-2 Alpha (54000 pfu). These mice were returned to their cages and their survival was monitored for 14 days as frank mortality. Between six- and eight-days following SARS-COV-2 Alpha dosing only 20% of these mice survived.

A second group of female hACE2 Knock-in mice was given an intranasal dose of SARS-CoV-2 Alpha (54000 pfu) followed by 300 micrograms of EVE18 24 hr, 48 hr and 72 hrs later. Mouse mortality was recorded for 10 days. EVE18 administration delayed mortality and enhanced survival in these mice to 50%.

A third group of female hACE2 Knock-in mice were given a sub-cutaneous dose of 300 micrograms of EVE18 2 hr prior to receiving SARS-COV-2 (54000 pfu) intranasally. These mice also received a second dose of intranasal EVE18 24 hr after virus. Pre- and early post-treatment of hACE2 mice with EVE18 delayed the mortality and enhanced the survival of this group of mice to 70%.

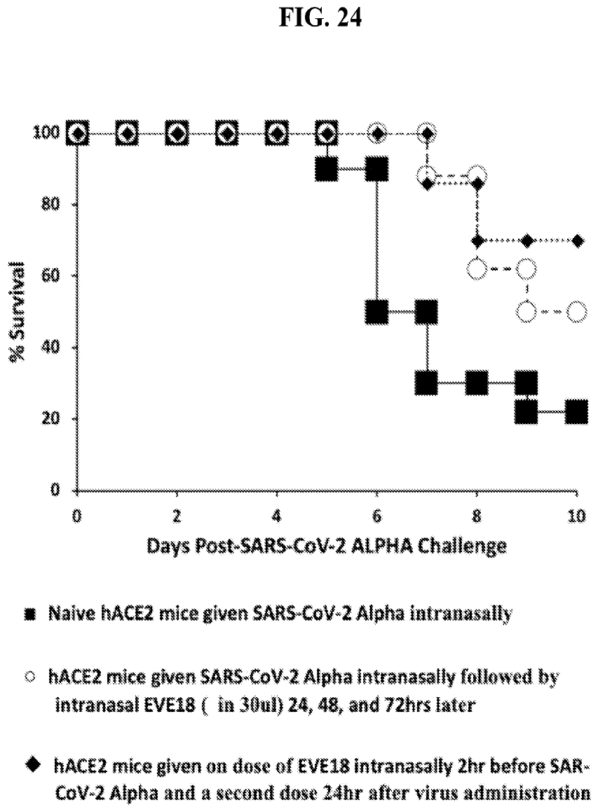

FIG. 24 demonstrates that EVE18 can reduce mortality in female hACE2 mice exposed to SARS-COV-2 Alpha when given in a therapeutic manner.

What is claimed:

1. A composition comprising an effective amount of a peptide, or a pharmaceutically acceptable form thereof, wherein the peptide comprises an amino acid sequence selected from the group consisting of: SEQ ID NOS: 1-39.

2. The composition of claim 1, further comprising at least one antiviral agent.

3. The composition of claim 1, wherein the peptide comprises the amino acid sequence of SEQ ID NO: 29.

4. The composition of claim 1, wherein the peptide comprises the amino acid sequence of SEQ ID NO: 30.

5. The composition of claim 1, wherein the peptide comprises the amino acid sequence of SEQ ID NO: 32.

6. The composition of claim 1, wherein the peptide comprises the amino acid sequence of SEQ ID NO: 33.

7. A method for treating an enveloped virus in a subject or in a pharmaceutical preparation, comprising administering to said subject or exposing an enveloped virus to an effective amount of (i) a peptide, or a pharmaceutically acceptable form thereof, wherein the peptide comprises a sequence selected from the group consisting of: SEQ ID NOS: 1-39, (ii) other cationic or zwitterionic amphipathic peptides, (iii) cationically charged membrane-active agents, (iv) molecules with hydrophobicity, charge and polarity distributions similar to the peptides of (i) or (v) chemical combinations of these four.

8. The method of claim 7, wherein the treatment is configured to increase the presentation of enveloped virus solution-exposed, surface-exposed and interfacial antigens to the subject in a manner that enhances the subject's systemic and mucosal immune response.

9. The method of claim 7, wherein the treatment is configured to invoke a broad immunogenic response in the subject in a manner that induces homologous protection against the virus exposed to peptide.

10. The method of claim 7, wherein the treatment is configured to invoke a broad immunogenic response in the subject in a manner that induces heterologous protection against viruses different from the virus exposed to peptide.

11. The method of claim 7, wherein the peptide comprises the amino acid sequence of SEQ ID NO: 1.

12. The method of claim 7, wherein the peptide comprises the amino acid sequence of SEQ ID NO: 2.

13. The method of claim 7, wherein the peptide comprises the amino acid sequence of SEQ ID NO: 3.

14. The method of claim 7, wherein the peptide comprises the amino acid sequence of SEQ ID NO. 7.

15. The method of claim 7, wherein the peptide comprises the amino acid sequence of SEQ ID NO. 16.

16. The method of claim 7, wherein the peptide comprises the amino acid sequence of SEQ ID NO. 22.

17. The method of claim 7, wherein the peptide comprises the amino acid sequence of SEQ ID NO: 29.

18. The method of claim 7, wherein the peptide comprises the amino acid sequence of SEQ ID NO. 30.

19. The method of claim 7, wherein the peptide comprises the amino acid sequence of SEQ ID NO. 32.

20. The method of claim 7, wherein the peptide comprises the amino acid sequence of SEQ ID NO. 33.

21. The method of claim 7, wherein the peptide comprises the amino acid sequence of SEQ ID NO. 34.

22. The method of claim 7, wherein the peptide comprises the amino acid sequence of SEQ ID NO. 35.

23. The method of claim 7, wherein the peptide comprises the amino acid sequence of SEQ ID NO. 36.

24. The method of claim 7, wherein the peptide comprises the amino acid sequence of SEQ ID NO. 37.

25. The method of claim 7, wherein the peptide comprises the amino acid sequence of SEQ ID NO. 38.

26. The method of claim 7, wherein the peptide comprises the amino acid sequence of SEQ ID NO. 39.

27. The method of claim 7, wherein the peptide comprises the L-enantiomer of the constituent amino acids.

28. The method of claim 7, wherein the peptide comprises the D-enantiomer of the constituent amino acids

29. The method of claim 7, wherein the peptide comprises a combination of the D- and L-enantiomers of the constituent amino acids.

30. The method of claim 7, wherein the subject is human.

31. The method of claim 7, wherein the subject is a livestock of commercial value.

32. The method of claim 7, wherein the subject is a wild or feral mammal, non-mammal, bird or insect.

33. The method of claim 7, wherein the subject is diagnosed with the viral infection.

34. The method of claim 7, wherein the subject is not infected with the enveloped virus.

35. The method of claim 7, wherein the subject is diagnosed with pulmonary disease, cardiovascular disease, diabetes mellitus, bacterial superinfection, sepsis syndrome, hypertension, chronic lung disease (inclusive of asthma, chronic obstructive pulmonary disease, and emphysema), chronic renal disease, chronic liver disease, immunodeficiency, an immunocompromised condition, neurologic disorder, neurodevelopmental, or intellectual disability.

36. The method of claim 7, wherein the enveloped virus is selected from the group consisting of: Herpesviridae, Poxviridae, Hepadnaviridae, Asfarviridae, Flaviviridae, Togoviridae, Coronaviridae, Orthomyxoviridae, Paramyxoviridae, Rhabdoviridae, Bunyaviridae, Filoviridae, and Retroviridae.

37. The method of claim 7, wherein the enveloped virus is SARS-COV-2.

38. The method of claim 7, wherein the enveloped virus is an influenza virus.

39. The method of claim 7, wherein the subject has the Long COVID syndrome.

40. The method of claim 7, wherein the administration of the compound or pharmaceutically acceptable form thereof comprises two or more doses.

41. The method of claim 7, wherein a disease or condition related to the enveloped virus is treated or prevented.

42. The method of claim 41, wherein the disease or condition is Guillain-Barre Syndrome, primary viral pneumonia, secondary bacterial pneumonia, inflammatory conditions, multisystem inflammatory syndrome, tissue damage, or organ failure, immune system over activation, or combinations of the foregoing.

43. The method of claim 42, wherein the inflammatory condition is Long COVID.

44. The method of claim 7, further comprising at least one additional antiviral agent.

45. The method of claim 7, in which exposing enveloped virus to agents disclosed herein inactivate the virus and allow the inactive virus or a pharmaceutically acceptable form to produce homologous or heterologous adaptive immunity in a subject.

46. The method of claim 7, wherein the administration is parenteral, pulmonary, intra-nasal, oral-gastric, buccal, or intravenous.

47. The method of claim 7, wherein the administration is in natural settings.

48. The method of claim 7, wherein the peptide or a peptide-virus admixture is contained in a food or food-like preparation palatable to the subject.

49. The method of claim 7, wherein the peptide or a peptide-virus admixture is administered in natural settings as a liquid.

50. The method of claim 7, wherein after administration of the peptide or treatment with an inactivated envelope virus mixture, an increase in viral load or the viral load of the enveloped virus is reduced in the subject.
